(19) United States
(12) Patent Application Publication
(10) Pub. No.: US 2022/0296526 A1
(43) Pub. Date: Sep. 22, 2022

(54) CANNABINOID COMPOSITIONS, METHODS OF MAKING SAME AND USES THEREOF

(71) Applicant: HEXO OPERATIONS INC., Ottawa (CA)

(72) Inventors: Francois Chouinard, Blainville (CA); Justin Conway, Carlgnan (CA)

(21) Appl. No.: 17/637,048

(22) PCT Filed: Aug. 20, 2020
(86) PCT No.: PCT/CA2020/051135

§ 371 Date: Feb. 21, 2022

Related U.S. Application Data
(60) Provisional application No. 62/889,276, filed on Aug. 20, 2019.

(51) Int. Cl. A61K 9/16; A61K 31/352; A61K 31/05; A61K 38/48

(52) U.S. Cl. CPC A61K 9/1694; A61K 9/1623; A61K 31/352; A61K 31/05; A61K 9/1652; A61K 9/1617; A61K 38/4873

(57) ABSTRACT

The present disclosure relates to cannabinoid compositions that are rehydratable and that upon such rehydration retain at least some of the properties of the original cannabinoid emulsions from which the cannabinoid composition was obtained. In particular, to cannabinoid compositions formed by spray drying of an emulsion, the composition comprising at least one cannabinoid, a carrier oil, one or more emulsifiers, and a sugar carrier, where the composition has a water activity that is less than about 0.5. The emulsion may include a nanoemulsion, a microemulsion, or both a nanoemulsion and a microemulsion. The present disclosure also relates to method of manufacturing same.

### CROSS-REFERENCE TO RELATED APPLICATION

The present application claims the benefit of U.S. provisional patent application Ser. No. 62/889,276 filed on Aug. 20, 2019. The contents of the above-referenced document are incorporated herein by reference in their entirety.

### TECHNICAL FIELD

The present disclosure generally relates to the field of cannabinoid compositions suitable for use in cannabis infused products (e.g., beverages, human or pet edibles, confectionaries, etc.), and specifically to cannabinoid compositions obtained by spray drying of cannabinoid emulsions. The present disclosure also relates to spray drying methods for obtaining such cannabinoid compositions.

### BACKGROUND

Cannabis infused beverages, human or pet edibles, and/or confectionaries are expected to grow in popularity due to the existing and/or expected legalization of these product forms in Canada and other countries (e.g., United States) globally. As a result, attention has turned to how to prepare industrial scale quantities of these products to meet consumer demands. One approach is to provide a concentrated pre-mix formulation of the cannabis extract that could be easily shipped to a manufacturer. The manufacturer would then dilute the concentrated pre-mix formulation into different product bases to form a large variety of different beverages (e.g., alcoholic, non-alcoholic), human (e.g., chewing gums, mints) or pet (e.g., pet food, pet chew) edibles and/or confectionaries (e.g., lozenges) ready for commercial sale and consumption.

A key challenge is to ensure that the cannabinoids are sufficiently solubilized in the concentrated pre-mix formulation all the way to the final cannabis infused products. Because cannabis formulations are typically highly lipophilic and have poor aqueous solubility, emulsification-based systems used for the solubilisation of concentrated pre-mix formulations have been described that are able to satisfy some or preferably all of the following requirements: (i) improved water solubility of the cannabinoids to maximize the consumable limits of the cannabis (e.g., regulatory limits of 10 mg of cannabis per beverage package for Canada), (ii) storage stability over the normal expected shelf-life (e.g., at least 6 months), (iii) transport stability over varying travel conditions (e.g., extreme temperatures, excessive agitation, etc.), (iv) clear physical appearance (for clear products) or no discoloration (for opaque products) and/or no adverse effects (e.g., ringing, creaming, etc.), and (v) pleasant organoleptic properties (e.g., pleasing taste and smell).

Cannabinoid compositions comprising such emulsification-based systems (i.e., cannabinoid emulsions) generally include droplets of a carrier oil containing solubilized cannabinoids, the droplets being dispersed throughout a continuous aqueous phase. As such, these cannabinoid emulsions may notably be characterized by a particle size distribution (PSD) of the droplets in the cannabinoid emulsion. It has been shown that the PSD of such cannabinoid compositions may be correlated to the absorption of cannabinoids in the organism once the cannabinoid compositions is ingested.

However, cannabinoid emulsions exhibit a liquid or substantially liquid form (e.g., a liquid, a slurry, etc.), and as such may not be readily suitable for use in all cannabis infused products. For example, such emulsions may be well-suited for use in cannabis-infused beverages, but less so for use in cannabis-infused human or pet edibles for which admixing with a formulation exhibiting a substantially dry form (e.g., a powder) may be preferable. Powders may also be easier to transport and formulate and exhibit a longer shelf-life than emulsions.

Spray drying is a conventional chemical process used to produce dry particulate solids (i.e., dry powders), from a variety of liquid or substantially liquid materials. Spray drying processes for producing powders are well-known and disclosed, for example, in U.S. Pat. Nos. 5,976,574, 5,985,248, 6,001,336, 6,051,256, 6,077,543, and 6,423,344 and PCT Publications WO 96/32149, WO 99/16419, WO 01/00312, WO 01/85136 and in WO 02/09669, which are each incorporated herein in their entirety by reference. Dry powders obtained using such processes may also be rehydrated. However, spray drying of cannabinoid emulsions using the processes described above may be detrimental to at least some of the properties of the rehydrated formulations so obtained, such that at least some of the properties of the rehydrated formulations obtained post-spray drying may be different from at least some of the properties of the original cannabinoid emulsions, including but not limited to the PSD of the droplets of carrier oil in which the cannabinoids are solubilized. This in turn may negatively impact the potential use of such rehydrated formulations in cannabis-infused beverages, human or pet edibles and confectionaries, as well as other properties generally related to the reconstitution of cannabinoid emulsions and the absorption of the cannabinoids in the organism once ingested.

Accordingly, there remains a need for cannabinoid compositions that do not exhibit at least some of the shortcomings described above, specifically cannabinoid compositions that are rehydratable and that upon such rehydration retain at least some of the properties of the original cannabinoid emulsions from which the cannabinoid composition was obtained, such as but not limited to the PSD of the droplets of carrier oil in which the cannabinoids are solubilized.

### SUMMARY

This Summary is provided to introduce a selection of concepts in a simplified form that are further described below in the Detailed Description. This Summary is not intended to identify key aspects or essential aspects of the claimed subject matter.

In accordance with one broad aspect, the present disclosure relates to a cannabinoid composition (also referred herein as “cannabinoid powder”) formed by spray drying of a cannabinoid emulsion, where the composition is rehydratable. Upon rehydration, the composition retains at least some of the properties of the cannabinoid emulsion prior to being spray dried.

For example, the rehydration test includes adding 0.1 g composition to 50 ml of water.

In accordance with another broad aspect, the present disclosure relates to a cannabinoid composition formed by spray drying of a microemulsion. The cannabinoid composition comprises at least one cannabinoid, a carrier oil, one or more emulsifiers and a sugar carrier. The cannabinoid composition has a water activity that is less than about 0.5. Upon rehydration of the composition in the rehydration test, the composition generates a nanoemulsion having a D₅₀<200 nm.

In accordance with another broad aspect, the present disclosure relates to a cannabinoid composition formed by spray drying of a microemulsion. The cannabinoid composition comprises at least one cannabinoid, a carrier oil, one or more emulsifiers and a sugar carrier. The cannabinoid composition has a water activity that is less than about 0.5. Upon rehydration of the composition in the rehydration test, the composition generates a nanoemulsion having a particle size distribution (PSD) that is within the range of 5 nm to 200 nm.

In accordance with another broad aspect, the present disclosure relates to a cannabinoid composition formed by spray drying of an emulsion. The cannabinoid composition comprises at least one cannabinoid, a carrier oil, one or more emulsifiers and a sugar carrier. The cannabinoid composition has a water activity that is less than about 0.5. The emulsion has a D₅₀<200 nm prior to spray drying and in presence of water, preferably <120 nm, more preferably <100 nm. Upon rehydration of the composition in the rehydration test, the composition generates a nanoemulsion having a D₅₀<200 nm.

In accordance with another broad aspect, the present disclosure relates to a cannabinoid composition formed by spray drying of an emulsion. The cannabinoid composition comprises at least one cannabinoid, a carrier oil, one or more emulsifiers and a sugar carrier. The cannabinoid composition has a water activity that is less than about 0.5. The emulsion has a particle size distribution PSD₁ prior to spray drying. Upon rehydration of the cannabinoid composition in the rehydration test, the cannabinoid composition generates an emulsion having a particle size distribution PSD₂ that does not change by more than about 200% relative to PSD₁.

In accordance with another broad aspect, the present disclosure relates to a cannabinoid composition formed by spray drying of an emulsion, the emulsion having a particle size distribution PSD₁ included in the range of 5 nm to 120 nm. The cannabinoid composition comprises at least one cannabinoid, a carrier oil, one or more emulsifiers and a sugar carrier. The cannabinoid composition has a water activity that is less than about 0.5. Upon rehydration in the rehydration test, the cannabinoid composition generates a nanoemulsion having a particle size distribution PSD₂ included within the range of 15 nm to 250 nm.

In a non-limiting embodiment, the herein described compositions may have one or more of the following features, in any combination:
upon rehydration of the composition in the rehydration test, the composition generates a nanoemulsion having a D₅₀<200 nm, or a D₅₀≤150 nm, a D₅₀≤120 nm, a D₅₀≤100 nm, or a D₅₀≤80 nm, or a D₅₀≤60 nm, or a D₅₀≤50 nm.
upon rehydration of the composition in the rehydration test, the composition generates a nanoemulsion having a D₉₀<200 nm, or a D₉₀≤150 nm, a D₉₀≤120 nm, a D₉₀≤100 nm, or a D₉₀≤80 nm, or a D₉₀≤60 nm, or a D₉₀≤50 nm.
the at least one cannabinoid includes tetrahydrocannabinol (THC), preferably Δ9-tetrahydrocannabinol or Δ8-tetrahydrocannabinol.
the at least one cannabinoid includes cannabidiol (CBD), preferably Δ2-cannabidiol.
the at least one cannabinoid includes tetrahydrocannabinol (THC), preferably Δ9-tetrahydrocannabinol or Δ8-tetrahydrocannabinol and includes cannabidiol (CBD), preferably Δ2-cannabidiol.
the one or more emulsifiers is a plurality of emulsifiers.
the plurality of emulsifiers comprises at least one ionic emulsifier, at least one non-ionic emulsifier, or a combination thereof.
the sugar carrier has a melting point above 95° C.
the sugar carrier is selected from the group consisting of lactose, cyclodextrin and mannitol.
the water activity (a_{w}) is within the range of 0.04≤aw≤0.3.
the emulsion prior to spray drying includes a plurality of emulsions.
the plurality of emulsions includes nanoemulsions, microemulsions, or both nanoemulsions and microemulsions.
the composition includes a plurality of particles. The particles have a PSD that may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less.
the composition includes a plurality of particles. The particles have a D₉₀ that may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less.
the composition includes a plurality of particles. The particles have a D₅₀ that may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less.
upon rehydration, the composition generates a plurality of nanoemulsions.

In accordance with another broad aspect, the present disclosure relates to method for obtaining a cannabinoid composition. The method comprises providing an emulsion including at least one cannabinoid, a carrier oil, and one or more emulsifiers. The method then includes mixing the emulsion with a sugar carrier to obtain a feed, and spray drying the feed to obtain the cannabinoid composition, the spray drying being performed under conditions such that the cannabinoid composition has a water activity which is less than about 0.5, and such that the upon rehydration of the cannabinoid composition in a rehydration test, the composition generates a nanoemulsion having a D₅₀<200 nm, the rehydration test being adding 0.1 g of the cannabinoid composition in 50 ml of water.

In accordance with another broad aspect, the present disclosure relates to method for obtaining a cannabinoid composition. The method comprises providing an emulsion including at least one cannabinoid, a carrier oil, and one or more emulsifiers. The method then includes mixing the emulsion with a sugar carrier to obtain a feed, and spray drying the feed to obtain the cannabinoid composition, the spray drying being performed under conditions such that the cannabinoid composition has a water activity which is less than about 0.5, and such that the upon rehydration of the cannabinoid composition in a rehydration test, the composition generates a nanoemulsion having a particle size distribution (PSD) that is within the range of from 5 nm to 200 nm, the rehydration test being adding 0.1 g of the cannabinoid composition in 50 ml of water.

In accordance with another broad aspect, the present disclosure relates to method for obtaining a cannabinoid composition. The method comprises providing an emulsion including at least one cannabinoid, a carrier oil, and one or more emulsifiers, the emulsion having a D₅₀<200 nm in presence of water. The method then includes mixing the emulsion with a sugar carrier to obtain a feed, and spray drying the feed to obtain the cannabinoid composition, the spray drying being performed under conditions such that the cannabinoid composition has a water activity which is less than about 0.5, and such that the upon rehydration of the cannabinoid composition in a rehydration test, the composition generates a nanoemulsion having a D₅₀<200 nm, the rehydration test being adding 0.1 g of the cannabinoid composition in 50 ml of water.

In accordance with another broad aspect, the present disclosure relates to method for obtaining a cannabinoid composition. The method comprises providing an emulsion including at least one cannabinoid, a carrier oil, and one or more emulsifiers. The method then includes mixing the emulsion with a sugar carrier to obtain a feed, and spray drying the feed to obtain the cannabinoid composition, the spray drying being performed under conditions such that the cannabinoid composition has a water activity which is less than about 0.5, and such that the upon rehydration of the cannabinoid composition in a rehydration test, the composition generates an emulsion having a particle size distribution PSD₂ which is within about 200% of a particle size distribution PSD₁ of the emulsion prior to spray drying in presence of water, the rehydration test being adding 0.1 g of the cannabinoid composition in 50 ml of water.

In accordance with another broad aspect, the present disclosure relates to method for obtaining a cannabinoid composition. The method comprises providing an emulsion including at least one cannabinoid, a carrier oil, and one or more emulsifiers, the emulsion having a PSD₁ included in the range of 5 nm to 120 nm in presence of water. The method then includes mixing the emulsion with a sugar carrier to obtain a feed, and spray drying the feed to obtain the cannabinoid composition, the spray drying being performed under conditions such that the cannabinoid composition has a water activity which is less than about 0.5, and such that the upon rehydration of the cannabinoid composition in a rehydration test, the composition generates a nanoemulsion having a particle size distribution PSD₂ included within the range of 15 nm to 250 nm, the rehydration test being adding 0.1 g of the cannabinoid composition in 50 ml of water.

In a non-limiting embodiment, the herein described method(s) may have one or more of the following features, in any combination:
the conditions include performing the spray drying in a spray dryer apparatus having an inlet temperature within the range of 120-220° C.
the spray dryer apparatus has an outlet temperature within the range of 75-115° C.
the PSD₂ is within about 100% of PSD₁ in the hydration test, preferably PSD₂ is within about 50% of PSD₁ in the hydration test, more preferably PSD₂ is within about 25% of PSD₁ in the hydration test.
the PSD₂ lies within the range of from 50 nm to 200 nm.
the PSD₂ is from 15 nm to 150 nm, or from 20 nm to 150 nm, or from 30 nm to 150 nm, or from 40 nm to 150 nm.
the at least one cannabinoid includes tetrahydrocannabinol (THC), preferably Δ9-tetrahydrocannabinol or Δ8-tetrahydrocannabinol
the at least one cannabinoid includes cannabidiol (CBD), preferably Δ2-cannabidiol.
the at least one cannabinoid includes tetrahydrocannabinol (THC), preferably Δ9-tetrahydrocannabinol or Δ8-tetrahydrocannabinol and includes cannabidiol (CBD), preferably Δ2-cannabidiol.
the one or more emulsifiers is a plurality of emulsifiers.
the plurality of emulsifiers comprises at least one ionic emulsifier, at least one non-ionic emulsifier, or both at least one ionic emulsifier and at least one non-ionic emulsifier.
the sugar carrier has a melting point above 95° C.
the sugar carrier is selected from the group consisting of lactose, cyclodextrin and mannitol.
the water activity (a_{w}) is within the range of 0.04≤aw≤0.3.
the emulsion prior to spray drying includes a plurality of emulsions.
the plurality of emulsions includes nanoemulsions, microemulsions, or both nanoemulsions and microemulsions.
upon rehydration, the composition generates an emulsion having a D₉₀<250 nm, D₉₀<240 nm, D₉₀<230 nm, D₉₀<220 nm, D₉₀<210 nm, D₉₀<200 nm, or a D₉₀≤150 nm, a D₉₀≤120 nm, a D₉₀≤100 nm, or a D₉₀<80 nm, or a D₉₀≤60 nm, or a D₉₀≤50 nm.
the sugar carrier may be included in a pre-spray drying composition. The pre-spray drying composition includes the sugar carrier solubilized in an aqueous composition.
the composition includes a plurality of particles. The particles have a PSD that may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less.
the composition includes a plurality of particles. The particles have a D₉₀ that may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less.
the composition includes a plurality of particles. The particles have a D₅₀ that may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less.
the feed may include one or more organic solvent.
upon rehydration, the composition generates a plurality of nanoemulsions

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1 shows a process for producing cannabis products in accordance with a non-limiting embodiment of the present disclosure;

FIG. 2 shows a process for spray-drying a cannabinoid emulsion in accordance with a non-limiting embodiment of the present disclosure; and

FIGS. 3A and 3B show exemplary atomizers that may be used in the process of FIG. 2, in accordance with a non-limiting embodiment of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

The present disclosure generally relates to cannabinoid compositions that are rehydratable and that upon such rehydration retain at least some of the properties of the original cannabinoid emulsions from which the cannabinoid composition was obtained. The present disclosure also generally relates to a spray-drying process for use with cannabinoid emulsions to obtain such cannabinoid compositions.

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

As used herein, terms of degree such as “about”, “approximately” and “substantially” mean a reasonable amount of deviation of the modified term such that the end result is not significantly changed. These terms may refer to a measurable value such as an amount, a temporal duration, and the like, and are meant to encompass variations of +/−0.1% of the given value, preferably +/−0.5%, preferably +/−1%, preferably +/−2%, preferably +/−5% or preferably +/−10%.

For the purpose of this specification, the term “cannabis product(s)” includes goods that are produced from cannabis or hemp, which include plant material, oils, resins, drinks, food additives, edibles, creams, aerosol sprays and vaporization substances, for example. The term “cannabis material(s)” includes cannabis plant material, which refers to plants or parts thereof, and/or materials that are derived from cannabis plant material and are intended for further processing to produce one or more cannabis products, as further described below.

As used herein, the term “cannabinoid” is generally understood to include any chemical compound that acts upon a cannabinoid receptor. Cannabinoids are commonly used for recreational purposes to produce physiological effects associated with a feeling of physical and/or emotional satisfaction. Cannabinoids can also be useful in the treatment and/or prophylaxis of a wide variety of diseases or conditions, such as pain, anxiety, inflammation, autoimmune diseases, neurological disorder, psychiatric disorder, malignancy, metabolic disorder, nutritional deficiency, infectious disease, gastrointestinal disorder, or cardiovascular disorder. Cannabinoids may also have application as neuroprotectants, for example, in limiting neurological damage following ischemic insults, such as stroke and trauma, or in the treatment of neurodegenerative diseases such as Alzheimer's disease, Parkinson's disease and HIV dementia. Cannabinoids for inclusion in the compositions of the present disclosure include phytocannabinoids (i.e., found in cannabis and some other plants) and synthetic cannabinoids (i.e., manufactured artificially).

Examples of suitable phytocannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC), Δ9-tetrahydrocannabionolic acid B (THCA-B), Δ9-tetrahydrocannabiorcol (THC-C1), tetrahydrocannabivarin (THCV), tetrahydrocannabivarinic acid (THCVA), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a,10a-tetrahydrocannabinol (Δ6a,10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least PCT Patent Application Pub. No. WO2017/190249 and U.S. Patent Application Pub. No. US2014/0271940, which are incorporated by reference in their entirety.

Cannabidiol (CBD) means one or more of the following compounds: Δ2-cannabidiol, Δ5-cannabidiol (2-(6-isopropenyl-3-methyl-5-cyclohexen-1-yl)-5-pentyl-1,3-benzenediol); Δ4-cannabidiol (2-(6-isopropenyl-3-methyl-4-cyclohexen-1-yl)-5-pentyl-1,3-benzenediol); Δ3-cannabidiol (2-(6-isopropenyl-3-methyl-3-cyclohexen-1-yl)-5-pentyl-1,3-benzenediol); Δ3,7-cannabidiol (2-(6-isopropenyl-3-methylenecyclohex-1-yl)-5-pentyl-1,3-benzenediol); Δ2-cannabidiol (2-(6-isopropenyl-3-methyl-2-cyclohexen-1-yl)-5-pentyl-1,3-benzenediol); Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ9-cis-tetrahydrocannabinol (cis-THC), Δ9-tetrahydrocannabinol (Δ9-THC), Δ9-tetrahydrocannabinolic acid A (THCA-A), Δ10-tetrahydrocannabinol (Δ10-THC), Δ9-tetrahydrocannabinol-C4, Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

Examples of suitable synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, quinolinyl esters, and combinations thereof.

The cannabinoid in the compositions of the present disclosure may be in an acid form or a non-acid form, the latter also being referred to as the decarboxylated form since the non-acid form can be generated by decarboxylating the acid form. Preferably, where reference is made to a specific cannabinoid, it will be understood that the cannabinoid is in the decarboxylated form.

The cannabinoid in the compositions of the present disclosure may be a single cannabinoid or may be a combination of two or more cannabinoids. In a non-limiting example, the cannabinoid in the compositions of the present disclosure is cannabidiol (CBD), tetrahydrocannabinol (THC), or a mixture thereof.

As is known in the art, various cannabinoids can be used in combination to achieve a desired effect in a user. Suitable mixtures of cannabinoids that can be used in the present disclosure include but are not limited to a mixture of tetrahydrocannabinol (THC), and cannabidiol (CBD). Certain specific ratios of cannabinoids may be useful to produce the feeling of physical and/or emotional satisfaction and/or may be useful in the treatment or management of specific diseases or conditions.

In some embodiments, the (w/w) ratio of the THC to the CBD is between about 1:1000 and about 1000:1. Preferably, the (w/w) ratio of THC to CBD in the composition may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1.

The compositions of the present disclosure may comprise the at least one cannabinoid in a concentration of from about 0.001 mg/mL to about 100 mg/mL, including any amount therebetween or any ranges therein; in a non-limiting example, the compositions may comprise from about 0.002 mg/mL to about 100 mg/mL, from about 0.1 mg/mL to about 75 mg/mL, or from about 0.1 mg/mL to about 50 mg/mL, including any amount therebetween or any ranges therein, of the at least one cannabinoid.

Cannabinoids for use in the present compositions may be obtained from any suitable source material including, but not limited to, cannabis or hemp plant material (e.g., flowers, seeds, trichomes, and kief) or manufactured artificially (for example cannabinoids produced in yeast, for example as described in WO WO2018/148848). The cannabis or hemp plant material may be provided in milled form or may be in the form of cannabis extracts obtained from cannabis or hemp plant material (e.g., resins, waxes and concentrates). As used herein, a “cannabis extract” refers to an extract obtained from a cannabis plant material according to any procedure known in the art; such extracts yield cannabinoids in substantially pure or isolated form. For example, a cannabis extract may be obtained by a process including an extraction step from plant materials using for example organic solvent extraction, such as extraction with CO2, butane, ethanol, and the like. For example, a cannabis extract may be obtained by a process including an extraction step from plant materials using for example heat decarboxylation to convert cannabinoids in their acid forms to neutral forms followed by or after CO₂ extraction (under sub-critical or super-critical conditions), providing a crude extract. The crude extract may then be “winterized”, that is, extracted with ethanol to remove lipids and waxes, as described for example in U.S. Pat. No. 7,700,368, US 2004/0049059, and US 2008/0167483, which are incorporated herein by reference. Optionally, the method for obtaining the cannabis extract may further include purification steps such as a distillation step to further purify, isolate or crystallize one or more cannabinoids, which is referred to herein as a “distillate”; US20160346339, which is incorporated herein by reference, describes a process for extracting cannabinoids from cannabis plant material using solvent extraction followed by filtration, and evaporation of the solvent in a distiller to obtain a distillate. The distillate may be further cut with one or more terpenes. The distillate may be further purified, for example using chromatographic and other separation methods known in the art, to obtain an “isolate”.

In some embodiments, pure or isolated cannabinoids, such as those provided in a cannabis extract, may be combined with water, lipids, hydrocarbons (e.g., butane), ethanol, acetone, isopropanol, or mixtures thereof.

Cannabinoid used in the compositions of the present invention may be an isolated cannabinoid, such as a cannabis extract, having >75% purity (as in the case of a crude extract), or >80% purity (as in the case of a distillate), or >95% purity, as in the case of an isolate). For example, and without wishing to be limiting, the cannabinoid may have >75%, preferably >80%, preferably >90%, preferably >95%, preferably >98%, preferably >98%, preferably >99% or preferably >99.5%, purity. It is especially preferred that the cannabinoids have high purity (i.e., Pharmacopoeia Grade substances, which may be obtained from a natural source or via synthetic means) to enable sufficient solubility in the composition once rehydrated.

### Recreational Products

Cannabinoids are commonly used for recreational purposes to produce physiological effects associated with a feeling of physical and/or emotional satisfaction. Cannabinoids can also be useful in the treatment and/or prophylaxis of a wide variety of diseases or conditions, such as pain, anxiety, inflammation, autoimmune diseases, neurological disorder, psychiatric disorder, malignancy, metabolic disorder, nutritional deficiency, infectious disease, gastrointestinal disorder, or cardiovascular disorder. The cannabinoids may also have application as neuroprotectants, for example, in limiting neurological damage following ischemic insults, such as stroke and trauma, or in the treatment of neurodegenerative diseases such as Alzheimer's disease, Parkinson's disease and HIV dementia.

As noted above, various cannabinoids and combinations thereof can be incorporated into the compositions of the present disclosure in varying amounts sufficient to achieve a desired effect in a user, such as a psychoactive effect, a physiological effect, or a treatment of a condition. The compositions of the present disclosure may achieve a psychoactive effect, a physiological effect, or a combination thereof, in a user. By “psychoactive effect”, it is meant a substantial effect on mood, perception, consciousness, cognition, or behavior of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of the cannabinoid at sufficient amounts to mediate the therapeutic effects.

Suitable examples of source material comprising cannabinoids include, but are not limited to, cannabis or hemp plant material (e.g., flowers, seeds, trichomes, and kief), milled cannabis or hemp plant material, extracts obtained from cannabis or hemp plant material (e.g., resins, waxes and concentrates), and distilled extracts or kief. In some embodiments, pure or isolated cannabinoids and/or source materials comprising cannabinoids may be combined with water, lipids, hydrocarbons (e.g., butane), ethanol, acetone, isopropanol, or mixtures thereof, as further described below.

Cannabinoids may be provided as compositions to form a large variety of cannabis infused products (e.g., beverages, human or pet edibles, confectionaries). To this end, cannabinoids present in cannabis plants should be extracted, concentrated and provided in suitable compositions for use in such cannabis infused products, as further described below.

### Cannabinoid Extract Production

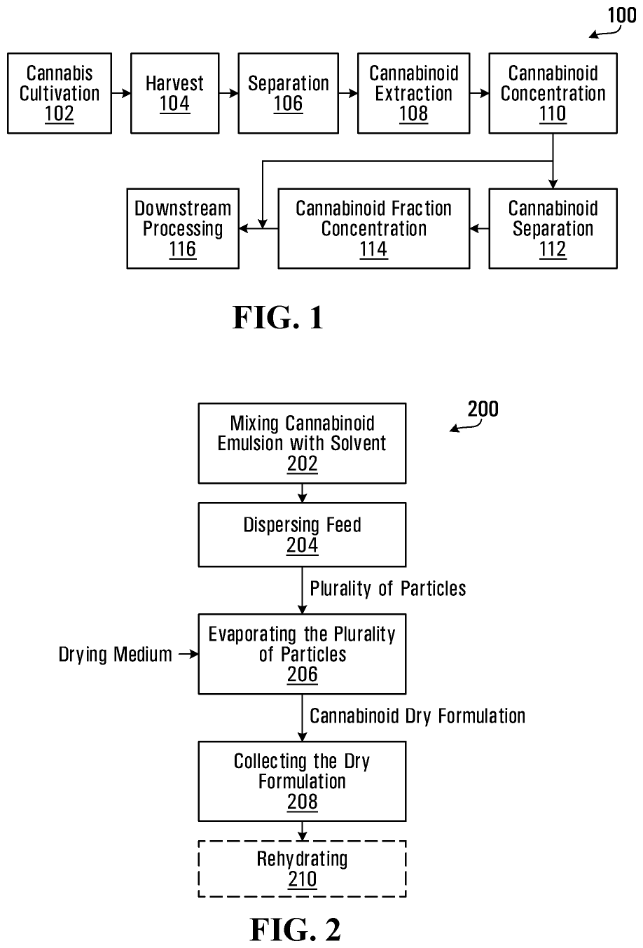

FIG. 1 shows a flow diagram illustrating a process 100 for processing cannabis material to produce a cannabinoid extract, in accordance with one non-limiting embodiment. At step 102, cannabis plants which contain cannabinoids are cultivated in a grow area and then are harvested at step 104 to obtain cannabis plant material. Cannabis plant material is intended to include any material that originates from a cannabis plant, including cannabis flowers, trims and/or waste for example. Cannabis flowers could also be referred to as buds and are typically harvested from mature cannabis plants. Trims includes the leaves of the cannabis plant that are separated from the flowers and stems. Trims could be harvested before the flowers, as the plants mature. Waste could include roots, stalks, stems and leaves that were not separated into trims, for example. The cannabis plant material obtained at the end of step 104 is then subjected to a separation step 106 that separates the cannabis plant material into distinct flower, trim and/or waste fractions. It will be readily appreciated that the separation step 106 could alternatively be performed on cannabis plant material that is supplied from a cannabis producer. In one non-limiting example, at step 106 the waste and the trims may be separated from the flowers.

The separated fraction obtained at the end of step 106 is then subjected to a cannabinoid extraction step 108. The purpose of the cannabinoid extraction step 108 is to solubilize the cannabinoids present in the separated fraction (e.g., flowers) using an extraction solvent to form a cannabinoid extract (i.e., a first cannabinoid composition). As such, the cannabinoid extraction step 108 is based at least in part on the solubility of the cannabinoids in an extraction solvent. Because cannabinoids are generally hydrophobic, hydrophobic solvents (in which the cannabinoids are soluble) are used as extraction solvents. The cannabinoid extraction step 108 includes processing or contacting the separated fraction with the extraction solvent, which separates the cannabinoids from the separated fraction and captures them in the form of an extract. Any material (which is not in the extract) that remains after the extraction is either treated as waste or subject to further processing. In some non-limiting examples, the extraction may be performed using any suitable (hydrophobic) solvent, such as but not limited to alcohol, hexane, propane, pentane, butane, acetone, and other hydrocarbons. In other non-limiting examples, the extraction solvent may be supercritical CO₂. Solvent-based extraction is one example of an extraction process. Mechanical extraction to separate trichomes, for example, may be used in other non-limiting embodiments. Other non-limiting embodiments could employ other types of extraction and/or multiple types of extraction, such as but not limited to liquid-liquid extraction, solid-phase extraction, solid-phase microextraction, Soxhlet extraction and fizzy extraction.

At step 110, the cannabinoid extract obtained at the end of step 108 is concentrated to increase the concentration of the extracted cannabinoids in the solvent to obtain a concentrated cannabinoid extract. In one non-limiting example, where a solvent extraction was used at step 108, the purpose of step 110 may be to eliminate at least a fraction of the solvent that was used at step 108 to reduce the volume of the cannabinoid extract obtained at the end of step 108 and therefore increase the concentration of the extracted cannabinoids in the solvent. In one non-limiting example, the concentration step may be performed using a rotary evaporator in which the solvent is removed from the extracted cannabinoid solution by evaporation. At step 112, the cannabinoids present in the concentrated cannabinoid extract obtained at the end of step 110 may then be separated to obtain distinct solubilized fractions each with a specific cannabinoid (e.g., one with THC, one with CBD, etc.). In one non-limiting example, the separation may be performed using high-performance liquid chromatography (HPLC) since distinct cannabinoids travel at different speeds through a suitable HPLC column (or stationary phase) such that they may each be eluted from the HPLC column at different times. At step 114, the specific cannabinoid fractions obtained at the end of step 112 may then be subjected to a further concentration step 114 generally similar to step 110 above (i.e., a rotary evaporator is used to remove at least a fraction of the solvent by evaporation). The resulting concentrated specific cannabinoid fractions may then be subjected to further downstream processing at step 116 to produce a variety of cannabis products, including cannabis infused products, as further described below. It will also be readily appreciated that, in some non-limiting embodiments, steps 112 and 114 may be omitted such that the concentrated extract obtained at the end of step 110 (which may contain a plurality of distinct cannabinoids) may be used directly as source material for the downstream processing at step 116. Any other process for processing cannabis material may be suitable in other non-limiting embodiments.

### Cannabinoid Emulsions

The concentrated cannabinoid extract obtained at the end of step 110 or the concentrated specific cannabinoid fractions obtained at the end of step 114 are compositions comprising one or more (hydrophobic) cannabinoids solubilized in a (hydrophobic) solvent, the cannabinoids having poor water aqueous solubility (i.e., being essentially water insoluble). For the subsequent use in cannabis infused products, in one non-limiting embodiment, aqueous compositions comprising cannabinoids ought to be produced, suitable examples of which include mixtures, suspensions or emulsions, preferably emulsions, even more preferably oil-in-water emulsions or microemulsions. An emulsion is termed an oil-in-water emulsion if the dispersed phase is an organic material and the continuous phase is water or an aqueous solution, or termed a water-in-oil emulsion if the dispersed phase is water or an aqueous solution and the continuous phase is an organic liquid (an “oil”). In one non-limiting embodiment, the concentrated cannabinoid extract obtained at the end of steps 110 or the concentrated specific cannabinoid fractions obtained at the end of step 114 may be subjected to a variety of downstream processing steps 116 so as to be provided as cannabinoid emulsions comprising a carrier oil or solvent (in which one or more cannabinoids are solubilized), one or more emulsifiers and the continuous phase, for example in the case of a nanoemulsion this would be an aqueous solution (e.g., water) and in the case of a microemulsion this would be an organic solvent (e.g., ethanol).

In one non-limiting example, the cannabinoid emulsion is a nanoemulsion that may exhibit a water content of at least about 10 wt. %, in some cases at least about 20 wt. %, in some cases at least about 30 wt. %, in some cases at least about 40 wt. % and in some cases even more. The water content as used herein means the total amount of water present in the cannabinoid emulsion, whether added separately or as a solvent or carrier for other raw materials.

In another non-limiting example, the cannabinoid emulsion is a microemulsion. This type of emulsion is a continuous system and contains mostly surfactant and little water. Typically, a microemulsion is not qualified by particle size as it is constantly in flux and multi-disperse. Generally, when a microemulsion is diluted into water, a nanoemulsion is released therefrom. In some embodiments, the microemulsion may exhibit a water content of less than 10 wt. %, less than 5 wt. %, less than 1 wt. %, or less than 0.5 wt. %. The microemulsion may include as continuous phase, for example, a non-aqueous solvent, such as ethanol, and the like.

In one non-limiting embodiment, the cannabinoid emulsion includes a single emulsion for example a nanoemulsion or a microemulsion.

In another non-limiting embodiment, the cannabinoid emulsion includes a plurality of emulsions such as a combination of nanoemulsions, a combination of microemulsions, or a combination of one or more nanoemulsion(s) and one or more microemulsion(s).

It will be readily appreciated that when the emulsion being spray dried includes a plurality of emulsions, the herein described composition upon rehydration may generate a corresponding plurality of emulsions. In other words, when the emulsion being spray dried includes an initial nanoemulsion and an initial microemulsion, the herein described composition upon rehydration may generate corresponding nanoemulsions (a first nanoemulsion corresponding to the initial nanoemulsion and a second nanoemulsion released from the initial microemulsion), where the first nanoemulsion has substantially identical particle size characteristics relative to that of the initial nanoemulsion and the second nanoemulsion has substantially identical particle size characteristics relative to that of the nanoemulsion that would be released from the initial microemulsion upon contacting water, as described herein. Similarly, when the emulsion being spray dried includes a plurality of initial nanoemulsions, each having respective particle size characteristics, the herein described composition upon rehydration may generate a corresponding plurality of nanoemulsions, each having substantially identical particle size characteristics relative to those of the initial nanoemulsions. Similarly, when the emulsion being spray dried includes a plurality of initial microemulsions, each being capable of releasing, in presence of water, nanoemulsions having respective particle size characteristics, the herein described composition upon rehydration may generate a corresponding plurality of nanoemulsions, each having substantially identical particle size characteristics relative to those which would be released from the microemulsions in presence of water.

The purpose of the carrier oil or solvent is to aid in solubilizing the hydrophobic cannabinoids in the emulsion. Non-limiting examples of suitable carrier oils or solvents include, but are not limited to, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (e.g., caprylic/capric glycerides), ethanol, acetone, isopropanol, hydrocarbons or a combination thereof.

The purpose of the one or more emulsifiers is to act as surfactants and to reduce a surface tension at an interface between the carrier oil and the aqueous solution. The one or more emulsifiers may be ionic, non-ionic or a combination of both. In some non-limiting examples, the emulsifier may be a polysorbate, polyoxyethylene, polyoxypropylene block co-polymer, ethoxylated aliphatic alkyl alcohol, ethoxylated fatty alcohol, ethoxylated aliphatic alkyl acid, ethoxylated fatty acid, glyceryl monostearate, sorbitan fatty acid ester, capril caprylic macrogolglycerides, propylene glycol laurate, propylene glycol caprylate, glycerol monostearate, polyglycerol oleate, lecithin-based emulsifier, tocopherol, polyoxyethylene or any combination therefore, and specifically polyoxyethylene monostearate (PEG 400 Monostearate), polyoxyethylene monooleate (PEG 400 Monoleate), polyoxyethylene sorbitan monolaurate (Tween® 20), polyoxyethylene sorbitan monolaurate (Tween® 21), polyoxyethylene sorbitan monopalmitate (Tween® 40), polyoxyethylene sorbitan monostearate (Tween® 60), polyoxyethylene sorbitan monostearate (Tween® 61), polyoxyethylene sorbitan tristearate (Tween® 65), polyoxyethylene sorbitan monooleate (Tween® 80), polyoxyethylene sorbitan monooleate (Tween 81), polyoxyethylene sorbitan trioleate (Tween® 85), polyoxyethylene-(15)-stearic acid (Pegosperse 1500MS), polyoxyethylene-(20)-stearyl alcohol (Brij 78), polyoxyethylene-(23)-lauryl alcohol (Brij 35), (Lutensol ON 60), PEG-40 hydrogenated castor oil (Cremophor/Kolliphor RH 40), PEG-35 castor oil (Cremophor EL), Solutol HS-15, sorbitan monopalmitate (Span 40), sorbitan monostearate (Span 60), sorbitan tristearate (Span 65), sorbitan monooleate (Span 80), sorbitan trioleate (Span 85), sunflower lecithin emulsifier, soybean lecithin emulsifier, linseed lecithin emulsifier, olive lecithin emulrapeseed lecithin emulsifier, egg lecithin emulsifier, corn lecithin emulsifier, peanut lecithin emulsifier, algal lecithin emulsifier, Vitamin E and Vitamin E derivatives (alpha, beta, gamma and delta-tocopherols), preferably d-alpha-tocopherol polyethyleneglycol 1000 succinate (Vitamin E TPGS), blend of isomers of alpha-tocopherol, beta-tocopherol, gamma-tocopherol and delta-tocopherol (Tocobiol), polyoxyethylene (2) cetyl ether (Brij C2), Quillaja extract (saponin), and any combination thereof. Any other suitable ionic or non-ionic emulsifier may be used in other non-limiting examples.

In some non-limiting examples, the emulsion is a nanoemulsion and includes one or more emulsifiers. In such cases, the one or more emulsifiers may be present in an amount of from about 0.1 wt. % to about 15 wt. %, in some cases from about 2 wt. % to about 12 wt. %, based on the total weight of the cannabinoid emulsion.

In other non-limiting examples, the emulsion is a microemulsion and includes one or more emulsifiers. In such cases, the one or more emulsifiers may be present in an amount of up to 85 wt. %, in some cases up to 80 wt. %, in some cases up to 75 wt. % based on the total weight of the cannabinoid emulsion.

In yet further non-limiting examples, the cannabinoid emulsion may operate to solubilize at least about 0.5 mg of cannabinoid in 1 mL of the emulsion, in some cases at least about 1 mg of cannabinoid in 1 mL of the emulsion, in some cases at least about 2 mg of cannabinoid in 1 mL of the of the emulsion, in some cases at least about 5 mg of cannabinoid in 1 mL of the of the emulsion, and in some cases even more.

In other non-limiting examples, the one or more emulsifiers may have a targeted combined HLB value that is equal to or greater than 11, preferably in the range of from 11 to 19, preferably in the range of from 11 to 17, or preferably in the range of from 11 to 15. As used herein, the term “targeted combine HLB value” refers to the HLB values which correspond not to a single emulsifier but the resulting HLB value of the blend of two or more emulsifiers needed to achieve a certain desired outcome. For example, it may be desirous to select a targeted combined HLB value for the one or more emulsifiers so that when it is formulated into an aqueous composition it operates in the solubilization of a certain level of cannabinoid containing extract (e.g., at least 1 mg of the cannabinoid in 1 mL of an aqueous composition). Although an exemplary level of at least 1 mg of the cannabinoid in 1 mL is provided herein, it is to be understood that it is desirable that higher levels of solubilized cannabinoids are possible with the emulsification system of the present disclosure (as further discussed below). The targeted combined HLB value contributes to better solubilization and stability of the cannabinoids in the emulsion. In this example the one or more emulsifiers may comprise: (i) at least one high HLB emulsifier, preferably a high HLB non-ionic emulsifier, having an individual HLB value of equal to or greater than 9, preferably in the range of from 9 to 17; and (ii) at least one low HLB emulsifier, preferably a low HLB non-ionic emulsifier, having an individual HLB value of below 9, preferably in the range of from 1 to 8.

In this non-limiting example, the high HLB non-ionic emulsifier may be selected from the group consisting of: polysorbates, polyoxyethylenes, polyoxypropylene block co-polymers, ethoxylated aliphatic alkyl alcohols, ethoxylated fatty alcohols, ethoxylated aliphatic alkyl acids, ethoxylated fatty acids, and a combination thereof. Suitable non-limiting examples of high HLB non-ionic emulsifier include one or more selected from the group consisting of: polyoxyethylene monostearate (PEG 400 Monostearate), polyoxyethylene monooleate (PEG 400 Monoleate), polyoxyethylene sorbitan monolaurate (Tween® 20), polyoxyethylene sorbitan monolaurate (Tween® 21), polyoxyethylene sorbitan monopalmitate (Tween® 40), polyoxyethylene sorbitan monostearate (Tween® 60), polyoxyethylene sorbitan monostearate (Tween® 61), polyoxyethylene sorbitan tristearate (Tween® 65), polyoxyethylene sorbitan monooleate (Tween® 80), polyoxyethylene sorbitan monooleate (Tween 81), polyoxyethylene sorbitan trioleate (Tween® 85), polyoxyethylene-(15)-stearic acid (Pegosperse 1500MS), polyoxyethylene-(20)-stearyl alcohol (Brij 78), polyoxyethylene-(23)-lauryl alcohol (Brij 35), (Lutensol ON 60), PEG-40 hydrogenated castor oil (Cremophor/Kolliphor RH 40), PEG-35 castor oil (Cremophor EL), Solutol HS-15 and a combination thereof, preferably polyoxyethylene sorbitan monooleate (Tween® 80). The high HLB non-ionic emulsifier may represent from about 55 wt. % to about 99 wt. % based on the total weight of the one or more emulsifiers.

Still in this example, the low HLB non-ionic emulsifier may be selected from the group consisting of glyceryl monostearates, sorbitan fatty acid esters, capril caprylic macrogolglycerides, propylene glycol laurates, propylene glycol caprylates, glycerol monostearate, polyglycerol oleates, lecithin-based emulsifiers, tocopherols, polyoxyethylenes, and a combination thereof. Suitable examples of low HLB non-ionic emulsifier include one or more selected from the group consisting of: sorbitan monopalmitate (Span 40), sorbitan monostearate (Span 60), sorbitan tristearate (Span 65), sorbitan monooleate (Span 80), sorbitan trioleate (Span 85), sunflower lecithin emulsifier, soybean lecithin emulsifier, linseed lecithin emulsifier, olive lecithin emulrapeseed lecithin emulsifier, egg lecithin emulsifier, corn lecithin emulsifier, peanut lecithin emulsifier, algal lecithin emulsifier, Vitamin E and Vitamin E derivatives (alpha, beta, gamma and delta-tocopherols), preferably d-alpha-tocopherol polyethyleneglycol 1000 succinate (Vitamin E TPGS), blend of isomers of alpha-tocopherol, beta-tocopherol, gamma-tocopherol and delta-tocopherol (Tocobiol), polyoxyethylene (2) cetyl ether (Brij C2), and a combination thereof. The low HLB non-ionic emulsifier may represent from about 1 wt. % to about 45 wt. % based on the total weight of the one or more emulsifiers.

In other non-limiting examples, when the emulsion is a nanoemulsion, a targeted one or more emulsifiers to carrier oil ratio may be from about 4.5:1 to about 3:1, in some cases from about 4.3:1 to about 3.2:1, however any other suitable ratio may be possible in other non-limiting examples.

In other non-limiting examples, when the emulsion is a microemulsion, a targeted one or more emulsifiers to carrier oil ratio may be from about 8:1 to about 3:1, however any other suitable ratio may be possible in other non-limiting examples.

As used herein, the term “targeted one or more emulsifiers to carrier oil ratio” refers to a measure of the level of the one or more emulsifiers to carrier oil that a formulator wishes to maintain so that when it is formulated into an aqueous composition it operates in the solubilisation of a certain level of cannabinoids (e.g., at least 1 mg of the cannabinoids in 1 mL of an aqueous solution). The term “ratio” refers to a mass ratio and the term “carrier oil” refers to the mass of the carrier oil.

In other non-limiting examples, when the emulsion is a nanoemulsion, a targeted carrier oil to water ratio may be from about 1:30 to about 1:40, preferably from about 1:33 to about 1:35, however any other suitable ratio may be possible in other non-limiting examples.

In other non-limiting examples, when the emulsion is a microemulsion, a targeted carrier oil to water ratio may be about 5:1, about 4:1, about 3:1, about 2:1, or about 1:1, since in some cases such emulsions can be mostly composed of surfactants.

As used herein, the term “targeted carrier oil to water ratio” refers to a measure of the level of carrier oil to water (as aqueous solution) in the emulsion that a formulator wishes to maintain so that when it is formulated into the aqueous composition it operates in the solubilisation of a certain level of cannabinoids (e.g., at least 1 mg of the cannabinoids in 1 mL of an aqueous solution). The term “ratio” refers to a mass ratio and the term “carrier oil” refers to the mass of the carrier oil.

The cannabinoid emulsion may be further characterized in several ways, for example a nanoemulsion can be characterized via its particle size distribution (PSD) and a microemulsion can be characterized via the PSD of the nanoemulsion released therefrom when the microemulsion is in presence of water. It will be readily appreciated that here, the term “cannabinoid emulsion” encompasses the cannabinoid emulsion prior to spray drying and/or the emulsion generated upon rehydration of the cannabinoid composition, which will be clear within the context of the text.

“Particle size distribution” or “PSD” is an index (means of expression) indicating what sizes (particle size) of particles are present in what proportions (relative particle amount as a percentage where the total amount of particles is 100%) in the sample particle group to be measured. Volume, area, length, and quantity are used as standards (dimensions) for particle amount. However, generally, the volume standard is used and expressed as the diameter of the particle. The PSD is usually determined over a list of size ranges that covers nearly all the sizes present in the tested sample. As used herein, the term “particle size” in respect of the emulsion refers to a volume-based particle size measured, for example, by dynamic light scattering (DLS) methods. It will be readily appreciated that the PSD of the cannabinoid emulsion refers to the PSD of the carrier oil droplets (as particles containing the solubilized cannabinoids) that are present in an aqueous solution. As such, in one non-limiting example, the cannabinoids may be encapsulated in the carrier oil droplets, the carrier oil droplets having a PSD in any range selected within the range of from about 5 nm to about 2000 nm.

For example, where a faster onset is desirable, the PSD may be included in the range from about 10 nm to about 200 nm, in some cases from about 20 nm to about 200 nm, in some cases from about 30 nm to about 200 nm, in some cases from about 40 nm to about 200 nm, in some cases from about 50 nm to about 200 nm, in some cases from about 60 nm to about 200 nm, in some cases from about 70 nm to about 200 nm, in some cases from about 80 nm to about 200 nm, in some cases from about 90 nm to about 200 nm, in some cases from about 100 nm to about 200 nm, or any PSD range there in between such as for example, from about 20 nm to about 120 nm, and the like.

For example, where a delayed onset may be desirable, the PSD may be included in the range from about 300 nm to about 2000 nm, in some cases from about 350 nm to about 2000 nm, in some cases from about 400 nm to about 2000 nm, in some cases from about 450 nm to about 2000 nm, in some cases from about 500 nm to about 2000 nm, in some cases from about 550 nm to about 2000 nm, in some cases from about 600 nm to about 2000 nm, in some cases from about 650 nm to about 2000 nm, in some cases from about 700 nm to about 2000 nm, in some cases from about 750 nm to about 2000 nm, or any PSD range there in between such as for example, from about 400 nm to about 800 nm, and the like.

It will be readily appreciated that the droplet size in the cannabinoid emulsion can be characterized with the average diameter of the carrier oil droplets (as particles containing the solubilized cannabinoids) that are present in the aqueous solution.

For example, the carrier oil droplets can be characterized as having a D₉₀ of 250 nm or less, in some cases about 240 nm or less, in some cases about 230 nm or less, in some cases about 220 nm or less, in some cases about 210 nm or less, in some cases about 200 nm or less, in some cases about 150 nm or less, in some cases about 120 nm or less, in some cases about 100 nm or less, in some cases about 80 nm or less, in some cases about 70 nm or less, in some cases about 60 nm or less, in some cases about 50 nm or less, in some cases about 40 nm or less, in some cases about 30 nm or less, in some cases about 20 nm or less, and in some cases even less. As used herein, the term “D₉₀” means the particle size value, defined as the hydrodynamic diameter of the particles of the dispersed phase, corresponding to the cumulative size distribution at 90%, which represents the size of particles below which 90% of the sample lies. For example, a D₉₀ of <200 nm means that 90% of the total amount of particles have a particle size of <200 nm.

For example, the carrier oil droplets can be characterized as having a D₅₀ of about 200 nm or less, in some cases about 150 nm or less, in some cases about 120 nm or less, in some cases about 100 nm or less, in some cases about 80 nm or less, in some cases about 70 nm or less, in some cases about 60 nm or less, in some cases about 50 nm or less, in some cases about 40 nm or less, in some cases about 30 nm or less, in some cases about 20 nm or less, and in some cases even less. The term “D₅₀” means the particle size value corresponding to the cumulative size distribution at 50%, which represents the size of particles below which 50% of the sample lies. For example, a D₅₀ of <100 nm means that 50% of the total amount of particles have a particle size of <100 nm.

The PSD of the cannabinoid emulsion may be controlled so as achieve a controlled onset and controlled offset of the cannabinoids once absorbed, or a fast or delayed onset of the cannabinoids once absorbed. “Absorption” of the cannabinoids may relate to any suitable mean of ingesting, applying or contacting the cannabinoid emulsion (i.e., a cannabis-infused edible or beverage containing the herein described cannabinoid emulsion) in or on a subject such that the cannabinoids contained within the cannabinoid emulsion are effectively absorbed by the subject. In one non-limiting example, the cannabinoid emulsion may have a PSD of from about 5 nm to about 200 nm to impart a fast onset of the cannabinoids, or any PSD range therein. Preferably, to impart the fast onset of the cannabinoids the cannabinoid emulsion has a PSD of from about 10 nm to about 80 nm, in some cases from about 10 nm to about 60 nm, in some cases from about 10 nm to about 40 nm, or any other value in-between.

In one non-limiting example, the controlled offset may be achieved by means of a distinct emulsion comprising at least one of an antidote, attenuator or modulator that modulates the absorption of the cannabinoids contained in the cannabinoid emulsion once absorbed. That is, much like the cannabinoid emulsion, the at least one antidote, attenuator or moderator is contained within droplets of carrier oil or solvent present in the aqueous solution. The antidote, attenuator or modulator emulsion has a PSD that is greater than the PSD of the cannabinoid emulsion such that the PSD of the antidote, attenuator or modulator emulsion imparts the delayed onset of the antidote, attenuator or moderator (i.e., the controlled offset of the cannabinoids). In some non-limiting examples, the antidote, attenuator or modulator emulsion may have a PSD which is within the range of from about 300 nm to about 2000 nm to impart the delayed onset of the antidote, attenuator or modulator once absorbed, in some cases from about 350 nm to about 2000 nm, in some cases from about 400 nm to about 2000 nm, in some cases from about 450 nm to about 2000 nm, in some cases from about 500 nm to about 2000 nm, in some cases from about 550 nm to about 2000 nm, in some cases from about 600 nm to about 2000 nm, in some cases from about 650 nm to about 2000 nm, in some cases from about 700 nm to about 2000 nm, in some cases from about 750 nm to about 2000 nm, and any PSD range there in between such as for example, from about 400 nm to about 800 nm, and the like. It will be readily appreciated that when both the cannabinoid emulsion and the antidote, attenuator or modulator emulsion are absorbed simultaneously, there is a differential absorption rate of the cannabinoid emulsion and the antidote, attenuator or modulator emulsion—i.e., the cannabinoid emulsion absorbing faster once ingested than the antidote, attenuator or modulator emulsion, thus resulting in a faster onset associated with the cannabinoids relative to the onset of the antidote, attenuator or modulator of the cannabinoids.

In one non-limiting example, the at least one antidote, attenuator or modulator may include one or more compound selected from cannabidiol (CBD), Acorus calamus or extracts thereof, black pepper or extracts thereof, citrus or extracts thereof, pine nuts or extracts thereof, pistachio nuts or extracts thereof, fruits of Pistacia terebinthus or extracts thereof, piperine, or terpenes, such as β-caryophyllene, limonene, myrcene, or α-pinene.

In other non-limiting examples, either one of the cannabinoid emulsion or the antidote, attenuator or modulator emulsion may further comprise at least one agent that further modulates the absorption of the cannabinoids contained in the cannabinoid emulsion once ingested, such as but not limited to a mucolytic, an efflux blocker, or any combinations thereof.

In yet further non-limiting examples, the cannabinoid emulsion may further comprise a liquid carrier, such as for example, water, preferably USP water. The water may be added as an ingredient on its own right or it may be present as a carrier in other common raw materials. In other non-limiting examples, the cannabinoid emulsion may include one or more other components such as, for example, a co-solvent, a preservative, or a buffering agent.

### Spray-Drying

FIG. 2 shows a non-limiting embodiment of a spray-drying process 200 for drying, or substantially drying, the cannabinoid emulsion described above. At step 102, the cannabinoid emulsion is mixed with a pre-drying composition for further processing, the mixture of the cannabinoid emulsion with the pre-drying composition being referred to as feed. The pre-drying composition may comprise (or in some cases, may consist of) a sugar carrier, which may be any suitable sugar, such as but not limited to lactose, polysaccharides such as maltodextrin and soy soluble polysaccharides, cyclodextrin, mannitol, gum arabic, starches such as corn starch, modified starches such as octenyl succinate modified starches, modified cellulose such as methyl cellulose, hydroxypropyl cellulose, methyl hydroxypropyl cellulose, and carboxymethylcellulose, certain types of pectin such as beet pectin, corn fiber gum and the likes. Preferably, the sugar carrier is selected from the group consisting of lactose, cyclodextrin and mannitol. In some embodiments, the pre-drying composition may include the sugar carrier in the form of a solubilized sugar carrier in a suitable medium, such as an aqueous composition and/or an organic solvent. Preferably, the suitable medium is water when the feed includes a nanoemulsion and the suitable medium is ethanol when the feed includes a microemulsion.

In some embodiments, the suitable sugar is one that has a melting point temperature that is suitable for use with the operating temperatures of the process described herein. For example, when one implements the process described herein in a spray-drying system, the spray-drying system may have operating parameters that include temperatures close to water boiling temperature or above, such as inlet and/or outlet temperature within the range of 100-220° C. In such practical implementations of the process described herein, the suitable sugar may thus have a melting point temperature that is suitable for such operating temperatures, such as for example a melting point temperature above 95° C., or above 100° C., or above 110° C., or above 120° C., or above 130° C., above 140° C., above 150° C., or more. In some embodiments, when the feed includes organic solvents, the spray-drying system may have operating parameters that include temperatures lower than water boiling temperature, and in these cases, the suitable sugar may thus have lower melting point temperature than the afore-mentioned values.

In one non-limiting example, a concentration of the sugar carrier in the pre-drying composition (in w/w) may be about 0.1%, in some cases about 0.5%, in some cases about 1%, in some cases about 1.5%, in some cases about 2%, in some cases about 2.5%, in some cases about 5%, in some cases about 10%, in some cases about 15%, in some cases about 20%, in some cases about 25%, in some cases about 50% and in some cases even more.

In another non-limiting example, a ratio of pre-drying composition to cannabinoid emulsion (in v/v) in the feed may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1.

In yet another non-limiting example, the feed may have a water content that is at least about 10 wt. %, in some cases at least about 20 wt. %, in some cases at least about 30 wt. %, in some cases at least about 40 wt. %, in some cases at least about 50 wt. %, in some cases at least about 60 wt. %, in some cases at least about 70 wt. % water and in some cases even more. Optionally, the feed may include one or ore organic solvents at various levels. The levels of such one or ore organic solvents may be reduced or the one or ore organic solvents may be completely removed when drying down the spray-dried composition.

Because the feed may generally be considered as being in a liquid or substantially liquid phase, the spray-drying process 200 will effect a transition from a liquid phase to a dry (or substantially dry) phase of the feed, the dry (or substantially dry) phase of the feed comprising a plurality of dried feed particles, as further described below.

In other non-limiting embodiments, additives may also be added to the feed for conformational stability during spray drying and for improving the dispersibility of the eventual cannabinoid composition. These additives include but are not limited to hydrophobic amino acids such as tryptophan, tyrosine, leucine, phenylalanine, and the likes. pH adjusters or buffers such as but not limited to organic salts prepared from organic acids and bases (e.g., sodium citrate, sodium ascorbate) and the likes may also be added to the feed in other non-limiting embodiments.

At step 204, the feed is dispersed by running the feed through an atomizer, dispersion referring to the process of atomizing the feed (comprising the cannabinoid emulsion mixed with the solvent) into a plurality of particles (or droplets). As such, the purpose of step 204 is to increase a surface area of the feed via the formation of the plurality of particles. Due to atomization there is an increase in the surface energy of the liquid, the magnitude of which is directly proportional to the increase of the surface area of the feed. The source of this energy increase depends on the type of atomizer used. The atomizer used at step 204 may be any suitably atomizer, such as but not limited to a pressure nozzle, a two-fluid nozzle, an ultrasonic nozzle, a rotary disc atomizer and the likes, the selection of a particular type of atomizer being reliant at least upon the nature and the amount of the feed being provided at step 102 as well as the desired characteristics of the eventual plurality of dried feed particles, which constitute the cannabinoid composition.

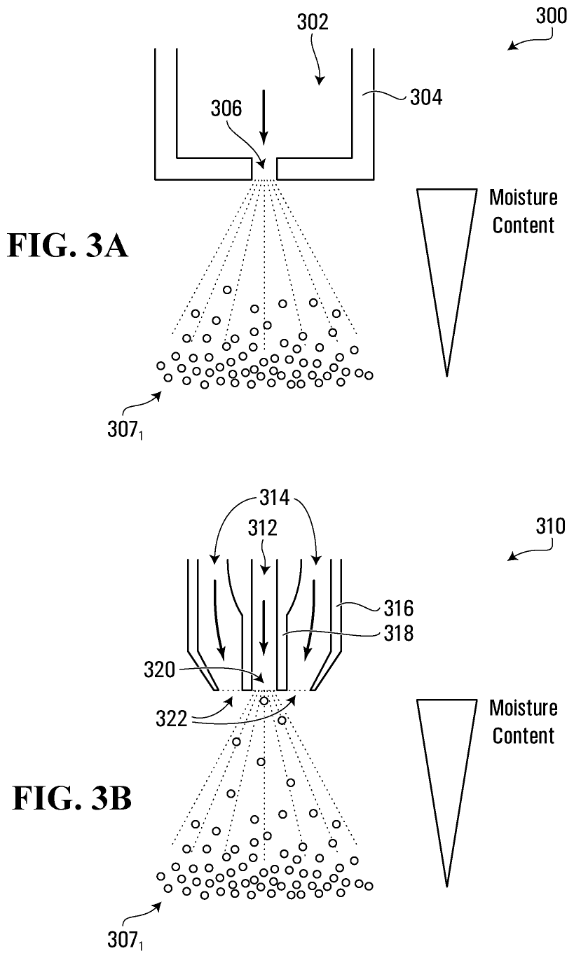

With further reference to FIG. 3A, a non-limiting example of a pressure nozzle 300 that may be used as atomizer at step 104 is provided. The pressure nozzle comprises an outer circumferential wall 304 defining an inner compartment 302 through which the feed is provided and an opening 306 through which the feed may exit the compartment 302 and be atomized in the plurality of particles 307ᵢ. Generally, the higher the pressure of the feed within the compartment 302, the higher the flow of the feed through the opening 306 and the smaller the size of the plurality of particles 307ᵢ at the exit of the opening 306. Smaller plurality of particles 307ᵢ at the exit of the opening 306 will ultimately result in smaller dried particles after the evaporating step 206, as further described below. Therefore, the pressure nozzle 300 atomizes the feed into the plurality of particles 307ᵢ by relying solely on the kinetic energy of the feed within the compartment 302. While the pressure nozzle 300 shown in FIG. 3A only exhibits a single opening 306, a plurality of openings may be also present and any other suitable configuration of the pressure nozzle 300 (e.g., geometrical configuration of the opening 306, presence of a surface impingement, pressure swirl, compound nozzles, etc.) may be suitable in other non-limiting examples.

With further reference to FIG. 3B, a non-limiting example of a two-fluid nozzle 310 that may be used as atomizer at step 104 is also provided. The two-fluid nozzle 310 comprises an outer circumferential wall 316 and an inner circumferential wall 318, the outer circumferential wall 316 and the inner circumferential wall 318 defining: (i) a first inner compartment 312 through which the feed is provided; (ii) a second inner compartment 314 through which a fluid, or atomization fluid, is provided; (iii) an opening 320 through which the feed may exit the compartment 312 and be atomized in the plurality of particles 307ᵢ and (iv) an opening 322 through which the atomization fluid may exit the compartment 314 and contact the feed as it exits the compartment 312. The pressure nozzle 310 atomizes the feed into the plurality of particles 307ᵢ by relying on the interaction between the flowing feed and the atomization fluid at the exit of the two-fluid nozzle 310. Much like the pressure nozzle 300 of FIG. 3A, the higher the pressure of the feed within the compartment 312, the higher the flow rate of feed through the opening 320 and the smaller the size of the plurality of particles 307ᵢ at the exit of the opening 306. Similarly, the higher the pressure of the fluid within the compartment 314, the smaller the size of the plurality of particles 307ᵢ at the exit of the opening 306. Again, smaller plurality of particles 307ᵢ at the exit of the opening 320 will ultimately result in smaller dried particles after the evaporating step 206, as further described below.

In the non-limiting example of FIG. 3B, the atomization fluid may be a gas such as compressed air, nitrogen or any other suitable gas and the gas is filtered or otherwise cleaned to remove particulates and other contaminants. The gas is pressurized for delivery through the opening 322, for example to a pressure of at least about 0.5 bar. While the two-fluid nozzle 300 of FIG. 3B has a configuration through which the feed and the atomization fluid are mixed externally to the two-fluid nozzle 300, any other suitable configuration is possible in other non-limiting examples (e.g., with internal mixing of the feed and fluid, etc.).

It will be readily appreciated that, as the plurality of particles 307ᵢ are formed at the exit of the atomizer, the plurality of particles 307ᵢ may not be dried or substantially dried, that is, the plurality of particles 307ᵢ may still retain a totality, a majority or a substantiality of the water content and water activity of the feed comprising the cannabinoid emulsion mixed with the solvent. At the exit of the atomizer, the water content and the water activity of the plurality of particles 307ᵢ may therefore be substantially identical to that of the feed being fed to the atomizer at step 204. The drying of the plurality of particles 307ᵢ is performed as the plurality of particles 307ᵢ travel away from the exit of atomizer at step 206, notably as the plurality of particles 307ᵢ come into contact with a drying medium within a chamber. The contact of the drying medium with the plurality of particles 307ᵢ will cause evaporation of the water present in the plurality of droplets 307ᵢ, as further described below.

In one non-limiting embodiment, the drying medium may be air, an inert gas such as nitrogen or any other suitable gas that has been filtered or otherwise treated to remove particulates and other contaminants. The drying medium is flowed through the plurality of particles 307ᵢ as they travel away from the atomizer up to a location where the particles will be collected, the flowing of the drying medium being performed using conventional blowers or compressors to move the drying medium from an inlet to an outlet. In some non-limiting examples, the flow rate of the drying medium via the inlet may be at least about 10 mL/hour, in some cases at least about 25 mL/hour, in some cases at least about 50 mL/hour, in some cases at least about 100 mL/hour, in some cases at least about 150 mL/hour, in some cases at least about 200 mL/hour, in some cases at least about 250 mL/hour, in some cases at least about 500 mL/hour, in some cases at least about 1000 mL/hour, in some cases at least about 2000 mL/hour and in some cases even more.

To cause evaporation of the solvent (e.g., water or organic solvent) present in the plurality of droplets 307ᵢ, the drying medium is heated and as such both the drying medium inlet and outlet temperatures should be controlled in order to control the residual solvent content (e.g., water activity) at the end of the spray drying process. That is, in some non-limiting examples, a temperature of the drying medium at the inlet may be at least about 80° C., in some cases at least about 100° C., in some cases at least about 120° C., in some cases at least about 140° C., in some cases at least about 160° C., in some cases at least about 170° C., in some cases at least about 180° C. and in some cases even more. For example, the temperature of the drying medium at the inlet may be within the range of temperatures of 120 to 220° C.

In other non-limiting examples, a temperature of the drying medium at the outlet may be at least about 50° C., in some cases at least about 60° C., in some cases at least about 70° C., in some cases at least about 80° C., in some cases at least about 90° C., in some cases at least about 100° C. and in some cases even more. For example, the temperature of the drying medium at the outlet may be within the range of temperatures of 75 to 115° C. It will be readily appreciated that the temperature of the drying medium at the outlet may be a function of at least the temperature of the drying medium at the inlet and the heat load imposed by the drying of the plurality of particles 307ᵢ (the heat load being itself a function of the temperature of the feed, the quantity of solvent to be evaporated in the feed and the likes).

It will be appreciated that when the feed includes one or more organic solvents, this may allow operating the spray drying procedure with lower temperatures than is the case when the feed includes water, which may be desirable in certain implementations.

In some embodiments, because the cannabis end-product is destined for human consumption, one may advantageously wish to avoid using organic solvents such as ethanol, propanol, butane, and the like, when preparing the feed thereby meeting growing consumer demand for eco-friendly technology and healthy formulations. It has been observed by the inventors that when implementing the herein described process in a spray-drying system, higher inlet temperatures are required when the feed does not include organic solvents. In such cases, it is thus desirable to use relatively higher inlet temperatures such as at least 100° C., or at least 110° C., or at least 120° C., at least 130° C., at least 140° C., at least 150° C., at least 160° C. and the like, which has an impact on the sugar carrier that is suitable for such operating parameters, as discussed previously.

In one non-limiting example, the drying medium may be flowed in substantially the same direction as the direction of travel of the plurality of particles 307ᵢ as they exit the atomizer. That is, in this example, the inlet is positioned in a vicinity of the atomizer and the plurality of particles 307ᵢ are contacted with a drying medium exhibiting a generally decreasing temperature profile as the plurality of particles 307ᵢ travel away from the atomizer. This results in vaporization, specifically evaporation, of the solvent present in the plurality of particles 307ᵢ, notably via evaporation of a saturated vapour film that is formed at a surface of the plurality of particles 307ᵢ, thereby forming the plurality of dried feed particles, which constitute the cannabinoid composition. In another non-limiting example, the drying medium may be flowed in a direction substantially opposite to the direction of travel of the plurality of particles 307ᵢ as they exit the atomizer. That is, in this example, the plurality of particles 307ᵢ are contacted with a drying medium exhibiting a generally increasing temperature profile as the plurality of particles 307ᵢ travel away from the atomizer. As the solvent evaporates from the plurality of particles 307ᵢ at step 206, that is as the solvent content and the water activity of the plurality of particles 307ᵢ decrease, the solubilized sugar carrier forms a sugar matrix around the droplets of carrier oil containing the solubilized cannabinoids that were originally present in the cannabinoid emulsion.

The chamber in which the plurality of particles 307ᵢ are contacted with the drying medium may be configured such that a residence time of the plurality of particles 307₁ within the chamber ensures drying of the plurality of particles 307ᵢ for example to a prescribed water content or water activity, while also ensuring that the plurality of particles 307ᵢ are collected before the temperature of the plurality of particles 307ᵢ rises to levels that could be damaging to either one of the sugar matrix formed during the drying process or the droplets of carrier oil. For the purpose of this specification the residence time may be defined as the time needed for the plurality of particles 307ᵢ to reach the prescribed solvent content and/or water activity. In one non-limiting example, the plurality of particles 307ᵢ are effectively dried (i.e., the drying has resulted in the plurality of dried feed particles) when the prescribed solvent content and/or water activity have been reached.

It will be readily appreciated that a drying rate of the plurality of particles 307ᵢ as they travel away from the atomizer may be dependent upon a plurality of parameters, such as but not limited to: (i) the overall surface area the plurality of particles 307ᵢ; (ii) the temperature of the plurality of particles 307ᵢ as they exit the atomizer; (iii) the temperature of the drying medium at the inlet and the outlet; (iv) the direction of flow of the drying medium relative to that of the plurality of particles 307ᵢ as they travel away from the atomizer; (y) the humidity of the drying medium; and (vi) the drying medium flow rate through the plurality of particles 307ᵢ.

At step 208, the plurality of dried feed particles so obtained may be collected in a collection device to obtain the cannabinoid composition. In one non-limiting example, the collection device may be a cyclone separator however conventional separation operations may also be used for example using a filter medium such as a membrane medium (bag filter), a sintered metal fiber filter and the likes. In some non-limiting examples, the cannabinoid composition may be subsequently packaged for further use, as further described below.

The cannabinoid composition, which includes the plurality of dried feed particles collected at step 208, may be characterized in a number of ways, including but not limited to by a PSD of the plurality of dried feed particles constituting the cannabinoid composition, a surface area of the plurality of dried feed particles, a rugosity of the plurality of dried feed particles, a water content and/or a water activity. In one non-limiting example, the PSD of the plurality of dried feed particles may be about 100 m or less, in some cases about 50 μm or less, in some cases about 25 μm or less, in some cases about 10 μm or less, in some cases about 5 μm or less, in some cases about 2 μm or less, in some cases about 1 μm or less and in some cases even less. In another non-limiting example, the D₉₀ of the plurality of dried feed particles may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less. In yet another non-limiting example, the D₅₀ of the plurality of dried feed particles may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less.

In yet another non-limiting example, the surface area of the plurality of dried particles per unit volume of feed may be at least about 0.5 m² per 100 mL of feed, in some cases at least about 1 m² per 100 mL of feed, in some cases at least about 2 m² per 100 mL of feed, in some cases at least about 3 m² per 100 mL of feed, in some cases at least about 4 m² per 100 mL of feed, in some cases at least about 5 m² per 100 mL of feed, in some cases at least about 10 m² per 100 mL of feed, in some cases at least about 15 m² per 100 mL of feed, in some cases at least about 20 m² per 100 mL of feed, in some cases at least about 25 m² per 100 mL of feed, in some cases at least about 50 m² per 100 mL of feed, in some cases at least about 100 m² per 100 mL of feed, in some cases at least about 150 m² per 100 mL of feed, in some cases at least about 200 m² per 100 mL of feed and in some cases even more.

As described above, it will be readily appreciated that the PSD of the plurality of dried feed particles may be dependent upon a number of parameters, including but not limited to: (i) the atomizer type; (ii) the operational parameters specific to the particular atomizer selected (e.g., feed pressure and flow, atomizing fluid pressure and flow, nozzle dimensions, opening size and numbers in the atomizer, atomizing fluid, etc.); (iii) the parameters of the evaporating step (e.g., drying medium type, drying medium temperature, drying medium humidity, drying medium flow direction, etc.); (iv) the mechanical properties of the feed (e.g., viscosity, etc.); and (v) the composition of the feed (e.g., solubilized sugar carrier concentration, type, etc.). The surface area of the plurality of dried feed particles may itself be dependent upon the PSD of the plurality of dried feed particles and the volume of feed subjected to dispersion at step 204. The larger the volume of feed being provided, the larger the surface area of the plurality of dried feed particles. Similarly, the smaller the PSD, the larger the surface area of the plurality of dried feed particles. To measure the PSD of the dry powder (dried feed particles), one can use any suitable technique known in the art. For example, the PSD can be measured by laser diffraction according to ISO 13320:2009 and ISO 9276-2:2014.

In other non-limiting examples, the cannabinoid composition may also be characterized by a rugosity of the plurality of dried particles, rugosity being a measure of surface convolution and being defined as the ratio between the actual surface of a particle and the geometrical surface of the particle (assuming for example that the particle is spherical), with a higher number indicating a higher degree of surface irregularity. Specifically, the cannabinoid composition may have a rugosity of at least about 1.3, in some cases at least about 1.4, in some cases at least about 1.5, in some cases at least about 1.6, in some cases at least about 1.7, in some cases at least about 1.8, in some cases at least about 1.9, in some cases at least about 2.0 and in some cases even more. It will be readily appreciated that an increase in the rugosity of the cannabinoid composition may be correlated with a decrease in the cohesiveness and surface interactions between adjacent particles of the plurality of dried feed particles. As such, an increase in the rugosity of the cannabinoid composition may be correlated with an increase in the dispersibility of the cannabinoid composition upon rehydration, as further described below. It will be further appreciated that the rugosity of the cannabinoid dried formulation may be dependent upon a variety of parameters, such as but not limited to: (i) the atomizer type; (ii) the operational parameters specific to the particular atomizer selected (e.g., feed pressure and flow, atomizing fluid pressure and flow, nozzle dimensions, opening size and numbers in the atomizer, atomizing fluid, etc.); (iii) the drying rate of the plurality of particles 307ᵢ as they travel away from the atomizer; and (iv) the composition of the feed.

In one non-limiting example, the cannabinoid composition may have a shelf-life (i.e., a storage length during which the cannabinoid composition does not become unfit for use and/or rehydration) that is at least about 1 month, in some cases at least about 2 months, in some cases at least about 3 months, in some cases at least about 6 months, in some cases at least about 1 year, in some cases at least about 2 years and in some cases even more.

Much like in the cannabinoid emulsion, the carrier oil droplets (in which the cannabinoids are solubilized) present in the emulsion generated upon rehydration of the cannabinoid composition of the present disclosure may also be characterized by a PSD.

In one non-limiting example, the carrier oil droplets present in the emulsion generated upon rehydration of the cannabinoid composition may exhibit a PSD₂ that is substantially identical to the PSD₁ of the carrier oil droplets present in the cannabinoid emulsion prior to spray drying or to the PSD₁′ of the carrier oil droplets that would be released from the cannabinoid microemulsion upon contacting an aqueous solution. In other words, the PSD₂ of the carrier oil droplets present in the emulsion generated upon rehydration of the cannabinoid composition does not change by more than about 200%, in some cases does not change by more than about 100%, in some cases does not change by more than about 50%, in some cases does not change by more than about 25%, in some cases does not change more than about 20%, in some cases does not change more than about 15%, in some cases does not change by more than about 10%, in some cases does not change by more than about 5%, in some cases does not change by more than about 2%, in some cases does not change by more than about 1% and in some cases even less compared to the PSD₁ of the carrier oil droplets present in the cannabinoid emulsion prior to being subjected to the spray drying process described above or to the PSD₁′ of the carrier oil droplets that would be released from the cannabinoid microemulsion upon contacting an aqueous solution. This stability in PSD can be tested, for example, in a rehydration test which includes adding 0.1 g of the composition in 50 ml of water.

In another non-limiting example, the carrier oil droplets present in the emulsion generated upon rehydration of the cannabinoid composition may exhibit a D₅₀ that is substantially identical to that of the carrier oil droplets present in the cannabinoid emulsion or to that one of the carrier oil droplets that would be released from the cannabinoid microemulsion upon contacting an aqueous solution. In other words, the D₅₀ of the carrier oil droplets present in the emulsion generated upon rehydration of the cannabinoid composition does not change by more than about 200%, in some cases does not change by more than about 100%, in some cases does not change by more than about 50%, in some cases does not change by more than about 25%, in some cases does not change more than about 20%, in some cases does not change more than about 15%, in some cases does not change by more than about 10%, in some cases does not change by more than about 5%, in some cases does not change by more than about 2%, in some cases does not change by more than about 1% and in some cases even less compared to the D₅₀ of the carrier oil droplets present in the cannabinoid emulsion prior to being subjected to the spray drying process described above or to the D₅₀′ of the carrier oil droplets that would be released from the cannabinoid microemulsion upon contacting an aqueous solution. This stability in D₅₀ can be tested, for example, in the afore-mentioned rehydration test.

In another non-limiting example, the carrier oil droplets present in the emulsion generated upon rehydration of the cannabinoid composition may exhibit a D₉₀ that is substantially identical to that of the carrier oil droplets present in the cannabinoid emulsion or to that of the carrier oil droplets that would be released from the cannabinoid microemulsion upon contacting an aqueous solution. In other words, the D₉₀ of the carrier oil droplets present in the emulsion generated upon rehydration of the cannabinoid composition does not change by more than about 200%, in some cases does not change by more than about 100%, in some cases does not change by more than about 50%, in some cases does not change by more than about 25%, in some cases does not change more than about 20%, in some cases does not change more than about 15%, in some cases does not change by more than about 10%, in some cases does not change by more than about 5%, in some cases does not change by more than about 2%, in some cases does not change by more than about 1% and in some cases even less compared to the D₉₀ of the carrier oil droplets present in the cannabinoid emulsion prior to being subjected to the spray drying process described above or to the D₉₀′ of the carrier oil droplets that would be released from the cannabinoid microemulsion upon contacting an aqueous solution. This stability in D₉₀ can be tested, for example, in the afore-mentioned rehydration test.

In yet another non-limiting example, the carrier oil droplets present in the emulsion generated upon rehydration of the cannabinoid composition may exhibit a PSD₂ that is substantially identical to the PSD₁ of the carrier oil droplets present in the cannabinoid emulsion or to the PSD₁′ of the carrier oil droplets that would be released from the cannabinoid microemulsion upon contacting an aqueous solution. For example, when the carrier oil droplets present in the cannabinoid emulsion, or the carrier oil droplets that would be released from the cannabinoid microemulsion upon contacting an aqueous solution, exhibit a PSD₁ that is within the range of from 5 nm to 200 nm, the carrier oil droplets present in the emulsion generated upon rehydration of the cannabinoid composition will exhibit a PSD₂ that is within the range of from 15 nm to 250 nm. This stability in PSD can also be tested, for example, in the afore-mentioned rehydration test.

In yet another non-limiting example, the carrier oil droplets present in the emulsion generated upon rehydration of the cannabinoid composition may exhibit a D₅₀ that is substantially identical to that of the carrier oil droplets present in the cannabinoid emulsion or to that of the carrier oil droplets that would be released from the cannabinoid microemulsion upon contacting an aqueous solution. For example, when the carrier oil droplets present in the cannabinoid emulsion prior to being subjected to the spray drying process described above, or the carrier oil droplets that would be released from the cannabinoid microemulsion upon contacting an aqueous solution, exhibit a D₅₀ that is <about 200 nm, the carrier oil droplets present in the emulsion generated upon rehydration of the cannabinoid composition will also exhibit a D₅₀ that is <about 200 nm. This stability in D₅₀ can also be tested, for example, in the afore-mentioned rehydration test.

In yet another non-limiting example, the carrier oil droplets present in the emulsion generated upon rehydration of the cannabinoid composition may exhibit a D₉₀ that is substantially identical to that of the carrier oil droplets present in the cannabinoid emulsion or to that of the carrier oil droplets that would be released from the cannabinoid microemulsion upon contacting an aqueous solution. For example, when the carrier oil droplets present in the cannabinoid emulsion prior to being subjected to the spray drying process described above, or the carrier oil droplets that would be released from the cannabinoid microemulsion upon contacting an aqueous solution, exhibit a D₉₀ which is <about 200 nm, the carrier oil droplets present in the emulsion generated upon rehydration of the cannabinoid composition will also exhibit a D₉₀ that is <about 200 nm. This stability in D₉₀ can also be tested, for example, in the afore-mentioned rehydration test.

In another non-limiting example, the PSD of the carrier oil droplets present in the emulsion generated upon rehydration of the cannabinoid composition, the D₉₀ of the carrier oil droplets present in the emulsion generated upon rehydration of the cannabinoid composition and/or D₅₀ of the carrier oil droplets present in the emulsion generated upon rehydration of the cannabinoid composition remain substantially identical after a storage period of at least about 1 month at 40° C., in some cases at least about 2 months at 40° C., in some cases at least about 6 months at 40° C., in some cases at least about 1 year at 40° C. and in some cases even more. That is, the PSD of the carrier oil droplets present in the emulsion generated upon rehydration of the cannabinoid composition, the D₉₀ of the carrier oil droplets present in the emulsion generated upon rehydration of the cannabinoid composition and/or D₅₀ of the carrier oil droplets present in the emulsion generated upon rehydration of the cannabinoid composition do not change by more than about 200%, in some cases do not change by more than about 100%, in some cases do not change by more than about 50%, in some cases do not change by more than about 25%, in some cases do not change by more than about 20%, in some cases do not change by more than about 15%, in some cases do not change by more than about 10%, in some cases do not change by more than about 5%, in some cases do not change by more than about 2%, in some cases do not change by more than about 1% and in some cases even less over the time periods described above.

In one non-limiting embodiment, the cannabinoid composition comprises an effective amount of cannabinoids for producing physiological effects associated with a feeling of physical and/or emotional satisfaction once formulated into the cannabis infused products (e.g., beverages, human or pet edibles, confectionaries). In another non-limiting embodiment, the cannabinoid composition comprises an effective amount of cannabinoids for treating or alleviating a disease or condition once formulated into the cannabis infused products (e.g., beverages, human or pet edibles, confectionaries). Preferably, the cannabinoid composition comprises the cannabinoid present in an amount of from about 1 mg/g of composition to about 50 mg/g of composition, in some cases from about 4 mg/g of composition to about 40 mg/g of composition, or in some cases from about 10 mg/g of composition to about 25 mg/g of composition. Cannabinoids provided at such an amount in the cannabinoid composition of the present disclosure can be particularly effective in delivering the desired physiological effects and/or treating or alleviating a disease or condition once formulated into the cannabis infused products. Such concentration of cannabinoid in the cannabinoid composition may also be effective in delivering a desired onset of action once formulated into the cannabis infused products.

In another non-limiting embodiment, the types of cannabinoids and/or the levels of the cannabinoids incorporated into the cannabinoid composition of the present disclosure provide substantially no psychoactive effect or no psychoactive effect. In other words, the types of cannabinoids and/or the levels of the cannabinoids used in the present cannabinoid composition do not substantially or do not affect mood, perception, consciousness, cognition or behavior of a subject, as a result of changes in the normal functioning of the nervous system.

In another embodiment, it is desirable that various cannabinoids can be used in combination to achieve the desired effect. Suitable combinations of the cannabinoid which can be used in the present disclosure include a combination of THC and CBD. Certain specific ratios of cannabinoids may be useful to produce the feeling of physical and/or emotional satisfaction and/or may be useful in the treatment or management of specific diseases or conditions.

In some non-limiting examples, the (w/w) ratio of the THC to the CBD is between about 1:1000 and about 1000:1. Preferably, the (w/w) ratio of THC to CBD in the cannabinoid composition may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1.

The cannabinoid composition described above may be suitable for use in cannabis infused products (e.g., beverages, human or pet edibles, confectionaries, etc.). That is, the cannabinoid composition may be added to a variety of beverages, human or pet edibles, confectionaries and the likes, as further described below. As used herein, the term “beverage” may comprise drinking water, juice, coffee, caffeinated beverage, tea, herbal tea, energy drink, non-alcoholic beverage (e.g., non-alcoholic beer), alcoholic beverage, cocoa beverage, distilled water, alkaline water, purified water, mineral water, coconut water, sparkling water, flavored water, natural fruit juice, synthetic fruit juice, natural vegetable juice, synthetic vegetable juice, carbonated drink, nitrogenated drink, beer, distilled spirit, lager, cider, wine/fortified wine and cocktail. As used herein, the term “edible” may comprise any product that is suitable, e.g., non-toxic, for placing into the mouth of a human or animal, whether ingested, absorbed, or only chewed or sucked on and at least a portion discarded, etc., examples of which include but are not limited to chewing or bubble gums, mints, suckers, jawbreakers, lozenges, hard candies, gummy candies, taffies, chocolates, brownies, cookies, crackers, granola or meal replacement bars, smokeless inhalation powders, honey, syrup, spreads, and dissolving strips. Preferably, the human edible products include gums, hard candies, soft candies, gummy candies, jellies, or lozenges, more preferably chewing or bubble gum or mints.

In some non-limiting examples, the cannabinoid composition may be used “as-is” in cannabis infused products, in which cases the cannabinoid composition may be provided directly in its powder form for addition/mixing in the cannabis infused product. It will be readily appreciated that in the instances in which the cannabis infused product is a beverage (e.g., an aqueous solution), the addition/mixing to the beverage will rehydrate the cannabinoid composition to form a rehydrated cannabinoid composition. In this case, the rehydrated cannabinoid composition results in the beverage comprising at least 1 mg of the cannabinoid per beverage package. As used herein, the term “beverage package” refers to a single unit of a beverage sold to consumer. For instance, a six-pack will constitute a single beverage package. Alternatively, a 1.5 L bottle of flavored soda will constitute a single beverage package. Therefore, if there are regulatory restrictions on the amount of cannabinoids (e.g., THC or CBD) permitted in a beverage package, it will have to be distributed evenly over the single beverage package. For example, at the time of this patent filing, Health Canada has proposed a maximum of 10 mg of THC per beverage package. This means a six-pack can contain less than 1.7 mg of THC per can or bottle. However, the levels of the cannabinoids in the beverage package are not necessarily limited and can conceivably be higher than the current approved regulatory limits, especially as the regulations change.

In one non-limiting example, the cannabinoid composition may be rehydrated in an aqueous solution (e.g., a beverage) thus generating an emulsion (e.g., a microemulsion and/or a nanoemulsion) that may exhibit a PSD that is substantially identical to that of the emulsion prior to the spray-drying process described above. That is the PSD of the carrier oil droplets present in the rehydrated cannabinoid composition does not change by more than about 200%, in some cases does not change by more than about 100%, in some cases does not change by more than about 50%, in some cases does not change by more than about 25%, in some cases does not change by more than about 20%, in some cases does not change by more than about 15%, in some cases does not change by more than about 10%, in some cases does not change by more than about 5%, in some cases does not change by more than about 2%, in some cases does not change by more than about 1% and in some cases even less compared to the PSD of the carrier oil droplets present in the cannabinoid emulsion that is subjected to the spray drying process described above.

In another non-limiting example, the carrier oil droplets present in the rehydrated cannabinoid composition may exhibit a D₉₀ that is substantially identical to that of the carrier oil droplets present in the cannabinoid emulsion that is subjected to the spray-drying process described above. That is the D₉₀ of the carrier oil droplets present in the rehydrated cannabinoid composition does not change by more than about 200%, in some cases does not change by more than about 100%, in some cases does not change by more than about 50%, in some cases does not change by more than about 25%, in some cases does not change by more than about 20%, in some cases does not change by more than about 15%, in some cases does not change by more than about 10%, in some cases does not change by more than about 5%, in some cases does not change by more than about 2%, in some cases does not change by more than about 1% and in some cases even less compared to the D₉₀ of the carrier oil droplets present in the cannabinoid emulsion that is subjected to the spray drying process described above.

In another non-limiting example, the rehydrated cannabinoid composition exhibits a PSD that is substantially identical to that of the cannabinoid emulsion as described above upon mixing of the cannabinoid composition with aqueous solutions exhibiting a variety of pH, salinity, carbonation levels and the likes. That is, in some non-limiting examples, the rehydrated cannabinoid composition exhibits a PSD that is substantially identical to that of the carrier oil droplets present in the cannabinoid emulsion as described above when the cannabinoid composition is rehydrated in an aqueous solution having a pH between about 4 and about 11, in some cases between about 5 and about 10, in some cases between about 6 and about 8 and in some cases between about 6.5 and 7.5 In other non-limiting examples, the rehydrated cannabinoid composition may also comprise one or more pH-adjusting agents to improve solubility and/or stability. It is believed that the pH modifiers can also aid with cannabinoid release during consumption and/or use. The pH of the rehydrated cannabinoid composition may be modified using any pharmaceutically acceptable means. Suitable examples of pH modifiers include, but are not limited to, organic acid or base, preferably tartaric acid, phosphoric acid, hydrochloric acid, maleic acid, sodium hydroxide, citric acid and the likes.

In yet further non-limiting examples, the rehydrated cannabinoid composition exhibits a PSD that is substantially identical to that of the carrier oil droplets present in the cannabinoid emulsion as described above when the cannabinoid composition is rehydrated in an aqueous composition having a salinity of less than about 40 g/kg, in some cases less than about 20 g/kg, in some cases less than about 10 g/kg, in some cases less than about 1 g/kg, in some cases less than about 500 mg/kg, in some cases less than about 250 mg/kg, in some cases less than about 125 mg/kg, in some cases less than about 100 mg/kg, in some cases less than about 50 mg/kg, in some cases less than about 25 mg/kg, in some cases less than about 10 mg/kg, in some cases less than about 1 mg/kg, in some cases less than about 0.1 mg/kg and in some cases even less.

In some non-limiting examples, a beverage cannabis-infused product as provided herein may be stable for at least about 1 month at 4° C., in some cases at least about 2 months at 4° C., in some cases at least about 3 months at 4° C., in some cases at least about 4 months at 4° C., in some cases at least about 5 months at 4° C., in some cases at least about 6 months at 4° C., in some cases at least about 7 months at 4° C., in some cases at least about 8 months at 4° C., in some cases at least about 9 months at 4° C., in some cases at least about 10 months at 4° C., in some cases at least about 11 months at 4° C., in some cases at least about 1 year at 4° C. and in some cases even more.

In other non-limiting examples, a cannabis-infused product provided herein may be stable for at least about 1 month at room temperature, in some cases at least about 2 months at room temperature, in some cases at least about 3 months at room temperature, in some cases at least about 4 months at room temperature, in some cases at least about 5 months at room temperature, in some cases at least about 6 months at room temperature, in some cases at least about 7 months at room temperature, in some cases at least about 8 months at room temperature, in some cases at least about 9 months at room temperature, in some cases at least about 10 months at room temperature, in some cases at least about 11 months at room temperature, in some cases at least about 1 year at room temperature and in some cases even more.

In other non-limiting examples, the cannabinoid composition may be used in human or pet edibles. The amount of the cannabinoids in the human edible product may be enough to produce a noticeable psychoactive effect associated with cannabinoids in a subject consuming at least a recommended amount of the edible product. Generally, a recommended amount is an amount that will produce psychoactive effects but not so great as to cause undesirable side effects or toxic effects.

In yet further non-limiting examples, the cannabinoid composition may be subjected to a variety of processing steps to form a variety of oral dosage forms for use in cannabis infused products, such as but not limited to tableting processes to form a variety of tablets (e.g., pills, caplets, etc.) for which at least a fraction of the tablets comprises the cannabinoid composition, coating processes to form a variety of coated particles in which at least a fraction of the coating comprises the cannabinoid composition, encapsulation processes in which at least the cannabinoid composition is encapsulated in a stable, hard shell, and the likes. It will be readily appreciated that in the above instances, rehydration of the cannabinoid composition may occur as the tablets, coated particles or capsules are ingested by an individual or as they are mixed with an aqueous solution. In yet further non-limiting examples, the cannabinoid composition may be rehydrated in an aqueous solution as it is subjected to at least some of the processing steps above. It will be readily appreciated that some of the processing steps described above may be used in the context of the production of human or pet edibles. For example, a human edible could be coated with the cannabinoid composition as described herein.

By properly controlling each one of the atomization, evaporation and collection steps above, the present disclosure enables the preparation of a cannabinoid composition produced by spray drying of a cannabinoid emulsion mixed with a solubilized sugar carrier, the cannabinoid composition being suitable for use in cannabis infused products and being rehydratable, the rehydrated cannabinoid composition retaining at least some of the characteristics of the cannabinoid emulsion.

In some non-limiting embodiments, the herein described procedures afford a cannabis-infused product which incorporates the cannabinoids in a stable manner. In other words, the cannabis-infused product advantageously remains stable in that there is close to no deterioration of the product appearance within the expected storage shelf-life.

### EXAMPLES

The following examples describe some exemplary modes of making and practicing certain compositions that are described herein. These examples are for illustrative purposes only and are not meant to limit the scope of the compositions and methods described herein.

### Example 1

In this example, microencapsulation compositions containing an emulsion having particle sizes >1000 nm (Formulation 1), 200 nm (Formulation 2) and 40 nm (Formulation 3) were made.

Cannabinoid based emulsions having a particle size of 40 nm and 200 nm are provided below in Tables 1 and 2. Cannabinoid based emulsions having a particle size of >1000 nm were prepared based on the formulae set out in Tables 1 and 2, without the additional sonication step. These exemplary formulations span the range from nano-emulsions to macro-emulsions. The foregoing emulsions were prepared as follows:
The water and oil phase ingredients were solubilized separately using heat and stirring. In particular, the water phase is comprised of water, Tween™ 80, ascorbic acid and EDTA and mixed at 60° C. with a magnetic stir bar for 30 minutes. The oil phase is comprised of Labrafac™ lipophile WL 1349, Tocobiol™, lecithin and THC distillate and mixed at 60° C. with a magnetic stir bar for 30 minutes.
Once the respective water and oil phases have been prepared they were combined while mixing with a high shear homogenizer at 8000-10000 rpm. The oil phase was added slowly to the water phase over 5 minutes and once completely the resultant emulsion was mixed for an additional 15 minutes. The resultant mixture is a macro-emulsion with a particle size >1000 nm.
To generate the 40 nm and 200 nm nano-emulsions, high energy sonication was applied to the macro-emulsions for 10 minutes with 100% amplitude using an LSP-500 Ultrasonic Processor (Sonomechanics, Florida, USA).

Using the same excipient components and tuning the ratio of emulsifiers to achieve the different particle sizes eliminates the experimental uncertainty in permeation data (see in later example) interpretation that would normally be associated if using different emulsifier combinations to achieve the different particle sizes.

Particle size of all nanoemulsions was measured in water solution at 25° C. using dynamic light scattering (DLS). All samples in the present disclosure have been analyzed at a dilution of 1/20 in purified water using a LiteSizer™ (Anton Paar GmbH, Germany).

TABLE 1

| Excipients | Mass (g) | wt. % |
|---|---|---|
| THC Distillate-03 | 18.75 | 2.5 |
| Labrafac lipophile | 20 | 2.67 |
| Ascorbic acid | 4.5 | 0.6 |
| Tocobiol | 3.75 | 0.5 |
| EDTA | 0.1 | 0.01 |
| Lecithin | 15 | 2 |
| Tween 80 | 60 | 8 |
| Water | 627.9 | 83.72 |

TABLE 2

| Excipients | Mass (g) | wt. % |
|---|---|---|
| THC Distillate-03 | 18.75 | 2.5 |
| Labrafac lipophile | 20 | 2.67 |
| Ascorbic acid | 3.75 | 0.5 |
| Tocobiol | 4.5 | 0.6 |
| EDTA | 0.1 | 0.01 |
| Lecithin | 10 | 1.33 |
| Tween 80 | 15 | 2 |
| Water | 677.9 | 90.39 |

The inventors discovered that different particle sizes of the emulsions were achieved by tuning the ratio of the plurality of emulsifiers present in the emulsification system. In particular, they found that a higher concentration of the high HLB value emulsifiers (e.g., Tween™ 80) relative to the low HLB value emulsifiers (e.g., Lecithin, Tocobiol™) generated smaller particle size (e.g., 40 nm) nano-emulsions. Conversely, the inventors discovered that a higher concentration of the low HLB value emulsifiers relative to the high HLB value emulsifiers resulted in the larger particle size (e.g., 200 nm) nano-emulsions. The results clearly demonstrate that the emulsification approach of the present disclosure allows for tuning the ratio of the emulsifiers to achieve different particle sizes suitable for formulating with a variety of product bases. Additionally, it eliminates the experimental uncertainty that would normally be associated with using different emulsifier combinations to achieve different particle sizes.

### Example 2

In this example, a microencapsulation composition containing THC with a particle size <100 nm was made.

1,000 mg of THC-containing cannabis oil was mixed with 50 mg of poly(ethylene glycol) monooleate with an appropriate amount of ethanol in a container to obtain an oil phase mixture. The oil phase mixture was heated at 50° C. until a liquid oil phase was obtained. In a separate container, 50 mg of sodium oleate were dissolved into 20 mL of deionized water to form an aqueous phase mixture. The oil phase mixture was added to the aqueous phase mixture and the combined mixture was mixed with a high shear mixer to obtain a coarse emulsion. A T25 (IKA, Staufen, Germany) at 8,000 rpm for 5 minutes can be used here. The coarse emulsion was mixed with a microfluidizer to further homogenize the emulsion and obtain the first microencapsulation composition containing THC with a particle size <100 nm. A Nano DeBEE homogenizer, (Westwood, Mass., USA) at 20,000 psi for 8-12 cycles can be used here.

### Example 3

In this example, a microencapsulation composition containing CBD with a PSD of about 200 nm was made.

5 g of limonene and 25 g of whey protein isolate were mixed with 70 g of water by stirring. The mixture was left for 24 hours to allow complete biopolymer hydration and saturation. After 24 hours, the mixture was homogenized using a sonicator. A Digital Sonifier 450 (Branson Ultrasonic Corporation, USA) at 160 W for 2 minutes can be used here. After homogenization, the emulsion was placed in an ice bath until the emulsion reached room temperature so as to obtain the second microencapsulation composition containing CBD with a PSD of about 200 nm.

### Example 4

In this example, a second microencapsulation composition containing CBD with a PSD of about 200 nm was made.

5 g of CBD-containing cannabis oil extract was mixed with 0.794 g Tween 80, 4.206 g Span 80, and 90 g distilled water in a test tube. The resulting mixture was heated to 70° C. and immediately homogenized to obtain the second microencapsulation composition containing CBD with a PSD of about 200 nm. An Ultra Turrax T 25 device (IKA, Staufen, Germany) at 13,400 rpm for 15 minutes can be used here.

### Example 5

In this example, a second microencapsulation composition containing CBD with a PSD of about 200 nm was made.

0.794 g Tween 80 was dissolved in 90 g distilled water to form an aqueous phase. 4.206 g Span 80 was dissolved in 5 g CBD cannabis oil to form an oil phase. Both the aqueous and oil phases were heated to 70° C. and maintained at this temperature.

The aqueous phase was added dropwise to the oil phase, while stirring the oil phase to obtain the second microencapsulation composition containing CBD with a PSD of about 200 nm. An RZR Heidolph homogenizer (Heidolph Instruments GmbH & Co. KG, Schwabach, Germany) at 1050 rpm over a duration of 30 min can be used here.

### Example 6

In this example, a second microencapsulation composition containing CBD with a PSD of about 200 nm was made. The same procedure as described in Example 5 was repeated except that 1.262 g Tween 80 was dissolved in 90 g distilled water to form the aqueous phase and 3.738 g Span 80 was dissolved in 5 g CBD cannabis oil extract to form the oil phase.

### Example 7

In this example, a second microencapsulation composition containing CBD with a PSD of about 200 nm was made.

The same procedure as described in Example 5 was repeated except that 1.729 g Tween 80 was dissolved in 90 g distilled water to form the aqueous phase and 3.271 g Span 80 was dissolved in 5 g CBD cannabis oil extract to form the oil phase.

### Example 8

In this example, a second microencapsulation composition containing CBD with a PSD of about 200 nm was made.

The same procedure as described in Example 5 was repeated except that 2.196 g Tween 80 was dissolved in 90 g distilled water to form the aqueous phase and 2.804 g Span 80 was dissolved in 5 g CBD cannabis oil extract to form the oil phase.

### Example 9

In this example, a second microencapsulation composition containing CBD with a PSD of about 200 nm was made.

The same procedure as described in Example 5 was repeated except that 2.664 g Tween 80 was dissolved in 90 g distilled water to form the aqueous phase and 2.336 g Span 80 was dissolved in 5 g CBD cannabis oil extract to form the oil phase.

### Example 10

In this example, a second microencapsulation composition containing CBD with a PSD of about 200 nm was made.

The same procedure as described in Example 5 was repeated except that 2.826 g Tween 80 was dissolved in 90 g distilled water to form the aqueous phase and 2.174 g Span 80 was dissolved in 5 g CBD cannabis oil extract to form the oil phase.

### Example 11

In this example, a second microencapsulation composition containing CBD with a PSD of about 200 nm was made.

The same procedure as described in Example 5 was repeated except that 3.370 g Tween 80 was dissolved in 90 g distilled water to form the aqueous phase and 1.630 g Span 80 was dissolved in 5 g CBD cannabis oil extract to form the oil phase.

### Example 12

In this example, a second microencapsulation composition containing CBD with a PSD of about 200 nm was made (Formulation K).

The same procedure as described in Example 5 was repeated except that 3.913 g Tween 80 was dissolved in 90 g distilled water to form the aqueous phase and 1.087 g Span 80 was dissolved in 5 g CBD cannabis oil extract to form the oil phase.

### Example 13—Mucolytic Agent

In this example, a microencapsulation composition containing THC and a mucolytic agent was made.

Kollipor EL (30% w/w) as surfactant and propylene glycol (47% w/w) as co-solvent were mixed with THC (3% w/w) at 40° C. for 30 minutes using a magnetic stirrer (Hotplate Stirrer Stuart) at the rate of 200 rpm. Captex 355 as oil (20% w/w) was added to this mixture and stirred for a further 30 min at 40° C. at 500 rpm. This mixture was dispersed in 0.1 M phosphate buffered saline solution (pH 6.8) with a volume ratio of 1:100 by stirring at 50 rpm. Papain-palmitate was dispersed in oleic acid at a concentration of 10% (m/v), and subsequently, equal volume of papain-palmitate dispersion and phosphate-buffered mixture were mixed at vortex for 10 min followed by sonication for 6 h at room temperature using Bandelin Sonorex at a frequency of 35 kHz. Droplet-sized particles were immediately observed after dispersing in 0.1 M phosphate buffer solution (pH 6.8) at a volume ratio of 1:100.

Papain-palmitate was prepared according to the following procedure:

Papain was dissolved in 0.1 M phosphate buffer (pH 8.0) at a concentration of 3 mg/ml using a thermomixer. Palmitoyl chloride solution in acetone at a concentration of 100 mg/ml was added dropwise into the papain solution at a volume ratio of 1:40. The pH was maintained at 8 by addition of 1 M NaOH. The reaction was conducted for 90 min at room temperature and produced a suspension. Afterwards, the modified papain suspension was dialyzed against water for 24 h followed by lyophilization.

This procedure for incorporating a mucolytic agent can be performed with any of the microencapsulation compositions described in the examples.

### Example 14—Efflux Blocker

In this example, a microencapsulation composition containing a cannabinoid and an efflux blocker was made.

504 mg of polysorbate 20, 504 mg of sorbitan monoleate, 504 mg of polyoxyl 40-hydroxy castor oil, and 504 mg of tricaprin were mixed in a container. In a separate container, 996 mg of ethyl lactate and 254 mg of lecithin were mixed and heated to 40° C. in a scintillation tube until complete dissolution. Both mixtures were mixed together using gentle stirring. The combined mixture was heated to 40° C. until a homogenous pre-concentrate solution was formed. 103 mg of cannabis oil was added to the pre-concentrate solution. The combined mixture was stirred gently, where upon gentle agitation of the cannabinoid in the aqueous phase, the pre-concentrate spontaneously forms drug encapsulated O/W nano-dispersion. 69 mg of an efflux blocker was added to form an advanced pro-nanoparticulates and the mixture was heated to 40° C. until a homogenous solution was formed.

This procedure for incorporating an efflux blocker can be performed with any of the microencapsulation compositions described in the examples.

### Example 15

In this example, various microencapsulation compositions containing THC at 2.5 wt. % were made in accordance with embodiments of the present disclosure and as per the procedure set forth in Example 1.

TABLE 3

| Ingredient | Mass (g) | wt. % |
|---|---|---|
| THC Distillate | 3.75 | 2.50 |
| Coconut Oil | 4 | 2.67 |
| Lecithin sunflower | 3 | 2 |
| Tween 80 | 12 | 8 |
| Water | 127.25 | 84.83 |

| | |
|---|---|
| PSD | 59.4 nm |

TABLE 4

| Ingredients | Mass (g) | wt. % |
|---|---|---|
| THC Distillate | 3.75 | 2.50 |
| Coconut Oil | 4.00 | 2.67 |
| Span 80 | 3.00 | 2.00 |
| Tween 80 | 12.00 | 8.00 |
| Water | 127.25 | 84.83 |

| | |
|---|---|
| PSD | 122.7 nm |

TABLE 5

| Ingredients | Mass (g) | wt. % |
|---|---|---|
| THC Distillate | 3.75 | 2.50 |
| Coconut Oil | 4.00 | 2.67 |
| Brij ™ C2-SO | 1.50 | 1.00 |
| Tween 80 | 11.00 | 7.33 |
| Water (g) : | 129.75 | 86.50 |

| | |
|---|---|
| PSD | 87.4 nm |

TABLE 6

| Ingredients | Mass (g) | wt. % |
|---|---|---|
| THC Distillate | 3.75 | 2.50 |
| Coconut Oil | 4.00 | 2.67 |
| Vit E TPGS | 3.00 | 2.00 |
| Tween 80 | 9.00 | 6.00 |
| Lecithin sunflower | 3.00 | 2.00 |
| Water | 127.25 | 84.83 |

| | |
|---|---|
| PSD | 36 nm |

TABLE 7

| Ingredients | Mass (g) | wt. % |
|---|---|---|
| THC Distillate | 3.75 | 1.74 |
| Vit E TPGS | 3.75 | 1.74 |
| Ethanol | 8.00 | 3.71 |
| Tween 20 | 150.00 | 69.61 |
| Water | 50.00 | 23.20 |

| | |
|---|---|
| PSD | 10 nm |

### Example 16

In this example, a nanoemulsion composition containing 50 mg/ml CBD was made in accordance with an embodiment of the present disclosure. The formulation for the nanoemulsion is shown in Table 8A.

TABLE 8A

| Ingredients | wt. % |
|---|---|
| CBD Distillate | 5.81 |
| Captex MCT | 2.76 |
| Ascorbic acid | 0.62 |
| Tocobiol | 0.52 |
| EDTA | 0.01 |
| Lecithin | 4.15 |
| Tween80 | 16.59 |
| Water (g): | 69.53 |
| Blend | 100.00 |

### Example 17

In this example, a microemulsion composition containing 5 wt. % CBD was made in accordance with an embodiment of the present disclosure. The formulation for the microemulsion is shown in Table 8B:

TABLE 8B

| | Quantity | |
|---|---|---|
| Ingredients | (g) | wt. % |
| Kolliphor RH 40 | 6.8 | 68 |
| CBD Isolate | 0.5 | 5 |
| MCT | 0.5 | 5 |
| Glyceryl Monooleate | 1.2 | 12 |
| (Peceol) | | |
| Water | 1 | 10 |
| Blend | 10 | 100 |

First glyceryl monooleate, CBD and MCT were combined into a first mixture, heated and solubilized together. Next, water and Kolliphor were combined into a second mixture, heated to 50 C and solubilized together. Finally, the first and second mixtures were combined and stirred to form a microemulsion. The microemulsion was characterized as having an average particle size of 30 nm, when dispersed in water with a PSD of 12%. Dispersion of the microemulsion in water spontaneously formed a nanoemulsion.

### Example 18

In this example, the nanoemulsion of Example 16 was spray dried as per an embodiment of the present disclosure.

The nanoemulsion was mixed with a solid sugar carrier using a stir bar at medium speed for 15 minutes before beginning the spray drying process. Prior to beginning the spray drying process, the Buchi 290 Spray Dryer (BUCHI Labortechnik AG, Germany) was assembled and set to the desired temperature. The machine was left to equilibrate for 30 minutes before starting the process. During this time water was pumped through the system.

Once the temperature was stabilized, the mixture of nanoemulsion and carrier was pumped into the Spray Dryer system via a feeding tube. Parameters were set on the instrument and the cannabinoid mixture was spray dried. The solution entered the drying chamber wherein the water was quickly evaporated, trapping the emulsion particles within the carrier matrix and creating a dried powder. The powder was directed through a cyclone chamber which funneled the material into the collection flask.

The following Table 8C lists the spray drying parameter ranges used. The parameters were selected based on the characterization post-spray drying with the main criteria being that re-hydrated samples are visually clear and particle size of the emulsion has been preserved.

TABLE 8C

| Parameter | Value |
|---|---|
| Inlet temp Range | 120-220° C. |
| outlet temp Range | 75-115° C. |
| Aspirator | 100% |
| Pump speed Range | 5-20% |

The resulting spray dried emulsion powder was analyzed by performing a rehydration and then performing a visual inspection for turbidity and DLS analysis for particle size tracking of the generated emulsion (see Table 9). The DLS measurements included: (1) nanoemulsion, (2) solubilized carrier, (3) pre-spray dried mixture of nanoemulsion and carrier, and (4) Re-hydrated nanoemulsion (0.1 g in 50 g water, diluted to obtain a clear signal with water).

Sorbitol was tested in several trial with a nanoemulsion having an average particle size of 54 nm where the feed did not include any organic solvent. It was observed that this sugar carrier failed in all of these trials—without being limited to any theory, it is believed that this failure was caused due to the high inlet temperatures required in the absence of organic solvents in the feed (i.e., the presence of an organic solvent in the feed allows one to use lower inlet temperatures), the melting point of sorbitol (95° C.) being below the spray drying parameters used when operating with a feed that is devoid of organic solvents. Accordingly, it was observed that using a sugar carrier having a melting point of at least 95° C., or at least 100° C., or at least 110° C., or at least 120° C., or at least 130° C., or at least 140° C., or at least 150° C., or at least 160° C., or at least 170° C., or at least 180° C., or at least 190° C., or at least 200° C., or at least 210° C., or at least 220° C., or more provided the desired result when operating with a feed that devoid of organic solvents.

TABLE 9

| Parameter | Measurement |
|---|---|
| Nanoemulsion | 0.025 g/ml |
| Kleptose | 0.4 g/ml |
| (Hydroxypropyl Cyclodextrin) | |
| Inlet temp | 120° C. |
| outlet temp | 75° C. |
| Aspirator | 100% |
| Pump speed | 5% |
| Particle Size Emulsion | 54 nm, clean signal |
| Particle Size Carrier | 2 nm, poor signal |
| Particle Size Emulsion + Carrier | 93 nm, clean signal, PDI 19.9% |
| Particle Size Rehydrated | 110 nm, clean signal, clear |
| | solution, PDI 19.7 |

TABLE 10

| Parameter | Measurement |
|---|---|
| Nanoemulsion | 0.025 g/ml |
| Pearlitol (Mannitol) | 0.4 g/ml |
| Inlet temp | 120° C. |
| outlet temp | 75° C. |
| Aspirator | 100% |
| Pump speed | 5% |
| Particle Size Emulsion | 54 nm, clean signal |
| Particle Size Carrier | 287 nm, poor signal |
| Particle Size Emulsion + Carrier | 80 nm, clean signal, PDI 17.4% |
| Particle Size Rehydrated | 81 nm, clean signal, clear |
| | solution, PDI 13.6 |

TABLE 11

| Parameter | Measurement |
|---|---|
| Nanoemulsion | 0.05 g/ml |
| Pearlitol (Mannitol) | 0.4 g/ml |
| Inlet temp | 120° C. |
| outlet temp | 75° C. |
| Aspirator | 100% |
| Pump speed | 5% |
| Particle Size Emulsion | 54 nm, clean signal |
| Particle Size Carrier | 225 nm, poor signal |
| Particle Size Emulsion + Carrier | 74 nm, clean signal, PDI 19.9% |
| Particle Size Rehydrated | 93 nm, clean signal, clear |
| | solution, PDI 19.7 |

TABLE 12

| Parameter | Measurement |
|---|---|
| Nanoemulsion | 0.05 g/ml |
| Maltodextrin | 0.4 g/ml |
| Inlet temp | 120° C. |
| outlet temp | 75° C. |
| Aspirator | 100% |
| Pump speed | 5% |
| Particle Size Emulsion | 54 nm, clean signal |
| Particle Size Carrier | 12 nm, poor signal |
| Particle Size Emulsion + Carrier | 118 nm, clean signal, PDI 19.9% |
| Particle Size Rehydrated | 130 nm, clean signal, clear |
| | solution, PDI 19.7 |

TABLE 13

| Parameter | Measurement |
|---|---|
| Nanoemulsion | 0.025 g/ml |
| Lactose Monohydrate | 0.4 g/ml |
| Inlet temp | 120° C. |
| outlet temp | 75° C. |
| Aspirator | 100% |
| Pump speed | 5% |
| Particle Size Emulsion | 54 nm, clean signal |
| Particle Size Carrier | 12 nm, poor signal |
| Particle Size Emulsion + Carrier | 70 nm, clean signal, PDI 19.9% |
| Particle Size Rehydrated | 136 nm, clean signal, clear |
| | solution, PDI 19.7 |

TABLE 14

| Parameter | Measurement |
|---|---|
| Nanoemulsion | 0.05 g/ml |
| Lactose Monohydrate | 0.4 g/ml |
| Inlet temp | 120° C. |
| outlet temp | 75° C. |
| Aspirator | 100% |
| Pump speed | 5% |
| Particle Size Emulsion | 54 nm, clean signal |
| Particle Size Carrier | 12 nm, poor signal |
| Particle Size Emulsion + Carrier | 72 nm, clean signal, PDI 19.9% |
| Particle Size Rehydrated | 153 nm, clean signal, clear |
| | solution, PDI 19.7 |

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art considering the present description that numerous modifications and variations can be made. The scope of the invention is defined more particularly in the appended claims.

1.-79. (canceled)

80. A cannabinoid composition in particle form formed by spray drying of an emulsion, the composition comprising:
a) at least one cannabinoid;
b) a carrier oil;
c) one or more emulsifiers; and
d) a sugar carrier,
wherein the composition has a water activity that is less than about 0.5,
wherein upon rehydration of the composition in a rehydration test, the composition generates a nanoemulsion having a D₅₀<200 nm, the rehydration test being adding 0.1 g of the composition in 50 ml of water.

81. The composition of claim 80, wherein the at least one cannabinoid includes tetrahydrocannabinol (THC).

82. The composition of claim 80, wherein the at least one cannabinoid includes cannabidiol (CBD).

83. The composition of claim 80, wherein the one or more emulsifiers comprises at least one ionic emulsifier, at least one non-ionic emulsifier, or a combination of at least one ionic emulsifier and at least one non-ionic emulsifier.

84. The composition of claim 80, wherein upon rehydration, the composition generates an emulsion having a D₅₀≤150 nm.

85. The composition of claim 80, wherein the sugar carrier has a melting point above 95° C.

86. The composition of claim 80, wherein the sugar carrier is selected from the group consisting of lactose, cyclodextrin and mannitol.

87. The composition of claim 80, wherein the water activity (a_{w}) is within the range of 0.04≤a_{w}≤0.3.

88. The composition of claim 80, wherein prior to spray drying, the emulsion in presence of water has a D₅₀<200 nm.

89. A method for obtaining a cannabinoid composition in particle form, comprising
a) providing an emulsion including at least one cannabinoid, a carrier oil, and one or more emulsifiers, the emulsion having a D₅₀<200 nm in presence of water,
b) mixing the emulsion with a sugar carrier to obtain a feed, and
c) spray drying the feed to obtain the cannabinoid composition, the spray drying being performed under conditions such that the cannabinoid composition has a water activity which is less than about 0.5, and such that upon rehydration of the cannabinoid composition in a rehydration test, the composition generates a nanoemulsion having a D₅₀<200 nm, the rehydration test being adding 0.1 g of the cannabinoid composition in 50 ml of water.

90. The method of claim 89, wherein the at least one cannabinoid includes tetrahydrocannabinol (THC).

91. The method of claim 89, wherein the at least one cannabinoid includes cannabidiol (CBD).

92. The method of claim 89, wherein the one or more emulsifiers comprises at least one ionic emulsifier, at least one non-ionic emulsifier, or a combination of at least one ionic emulsifier and at least one non-ionic emulsifier.

93. The method of claim 89, wherein the D₅₀ of the emulsion in step a) in presence of water is s 120 nm.

94. The method of claim 89, wherein the sugar carrier has a melting point above 95° C.

95. The method of claim 89, wherein the sugar carrier is selected from the group consisting of lactose, cyclodextrin and mannitol.

96. The method of claim 89, wherein the water activity (a_{w}) is within the range of 0.04≤a_{w}≤0.3.

97. The method of claim 89, wherein the emulsion in step a) includes a plurality of emulsions.

98. The method of claim 97, wherein the plurality of emulsions includes nanoemulsions, microemulsions, or both nanoemulsions and microemulsions.

99. The method of claim 89, wherein the conditions include performing the spray drying in a spray dryer apparatus having an inlet temperature within the range of 120-220° C.

100. The method of claim 89, wherein the spray dryer apparatus has an outlet temperature within the range of 75-115° C.
